(19) United States
(12) Patent Application Publication
(10) Pub. No.: US 2021/0171971 A1
(43) Pub. Date: Jun. 10, 2021

(54) INTERACTORS AND TARGETS FOR IMPROVING PLANT AGRONOMIC CHARACTERISTICS

(71) Applicant: PIONEER HI-BRED INTERNATIONAL, INC., JOHNSTON, IA (US)

(72) Inventors: KRISTIN HAUG COLLET, DES MOINES, IA (US); ZHENGLIN HOU, ANKENY, IA (US); SHAI LAWIT, URBANDALE, IA (US); ROSANA MELO, ST. CHARLES, MO (US); NICHOLAS MONGAR, JOHNSTON, IA (US); JOHN VAN HEMERT, JOHNSTON, IA (US); JINGRUI WU, JOHNSTON, IA (US); WENGANG ZHOU, SAN MATEO, CA (US)

(73) Assignee: PIONEER HI-BRED INTERNATIONAL, INC., JOHNSTON, IA (US)

(21) Appl. No.: 17/048,038

(22) PCT Filed: Apr. 16, 2019
(86) PCT No.: PCT/US19/27617

§ 371 Date: Oct. 15, 2020

Related U.S. Application Data
(60) Provisional application No. 62/778,086, filed on Dec. 11, 2018.
(60) Provisional application No. 62/741,529, filed on Oct. 4, 2018.
(60) Provisional application No. 62/659,579, filed on Apr. 18, 2018.

(51) Int. Cl. C12N 15/82

(52) U.S. Cl. CPC C12N 15/8261; C12N 15/8271; C12N 15/8213; C12N 15/8269

(57) ABSTRACT

Provided are compositions comprising polynucleotides encoding polypeptides. Also provided are recombinant DNA constructs, plants, plant cells, seed, grain comprising the polynucleotides, and plants, plant cells, seed, grain comprising a genetic modification at a genomic locus encoding a polypeptide. Additionally, various methods of employing the polynucleotides and genetic modifications in plants, such as methods for modulating expression level in a plant and methods for increasing yield of a plant are also provided herein.

### REFERENCE TO SEQUENCE LISTING SUBMITTED ELECTRONICALLY

The official copy of the sequence listing is submitted electronically via EFS-Web as an ASCII formatted sequence listing with a file named 7841_ST25.txt created on Dec. 7, 2018 and having a size of 1897 kilobytes and is filed concurrently with the specification. The sequence listing comprised in this ASCII formatted document is part of the specification and is herein incorporated by reference in its entirety.

### FIELD

This disclosure relates to compositions and methods for improving yield in plants.

### BACKGROUND

Global demand and consumption of agricultural crops is increasing at a rapid pace. Accordingly, there is a need to develop new compositions and methods to increase yield in plants. This invention provides such compositions and methods.

### SUMMARY

Provided herein are polynucleotides encoding a polypeptide comprising an amino acid sequence that is at least 90%, 92%, 95%, 96%, 97%, 98%, 99% or 100% identical to a full length amino acid sequence selected from the group consisting of SEQ ID NOS: 1-11, 23-31, 40-299, 563, 565, and 567-573. Provided herein are polynucleotides that are at least 85% identical to a polynucleotide sequence selected from the group consisting of SEQ ID NOS: 12-22, 32-39, and 300-561.

Provided herein are SEQ ID NOS: 1-11 (Interactor Polypeptides); SEQ ID NOS: 12-22 (polynucleotides encoding Interactor Polypeptides); SEQ ID NOS: 23-31, 563, and 567-572 (Direct Target Polypeptides); SEQ ID NOS: 32-39, 564, and 573-579 (polynucleotides encoding Direct Target Polypeptides); SEQ ID NOS: 40-299 (Differentially Expressed Polypeptides); SEQ ID NOS: 300-561 (polynucleotides encoding Differentially Expressed Polupeptides); SEQ ID NOS: 223-284 (Down Regulated Polypeptides); SEQ ID NOS: 485-547 (polynucleotides encoding Down Regulated Polypeptides).

Also provided are recombinant DNA constructs comprising a regulatory element operably linked to a polynucleotide encoding a polypeptide comprising an amino acid sequence that is at least 80% to 100% identical to a full length amino acid sequence selected from the group consisting of SEQ ID NOS: 1-11, 23-31, 40-299, 563, 565, and 567-573. In certain embodiments the regulatory element is a heterologous promoter.

Also provided are recombinant DNA constructs comprising a genetic element that suppresses or reduces expression of a polynucleotide encoding a polypeptide comprising an amino acid sequence that is at least 80% to about 100% identical to an amino acid sequence selected from the group consisting of SEQ ID NOS: 223-284 (Down Regulated Polypeptides). In certain embodiments the reduction in expression is performed by RNAi mechanism.

Provided are plant cells, plants, and seeds comprising the polynucleotide encoding a polypeptide or the recombinant DNA construct comprising a regulatory element operably linked to the polynucleotide encoding a polypeptide. In certain embodiments, the regulatory element is a heterologous promoter. In certain embodiments, the plant and/or seed is from a monocot plant. In certain embodiments, the plant is a monocot plant. In certain embodiments, the monocot plant is maize.

Further provided are plant cells, plants, and seeds comprising a targeted genetic modification at a genomic locus that encodes a polypeptide comprising an amino acid sequence that is at least 80% to about 100% identical to a full length amino acid sequence selected from the group consisting of SEQ ID NOS: 1-11, 23-31, 40-299, 563, 565, and 567-573, wherein the genetic modification increases the level and/or activity of the encoded polypeptide. In certain embodiments, the genetic modification is selected from the group consisting of an insertion, deletion, single nucleotide polymorphism (SNP), and a polynucleotide modification. In certain embodiments the targeted genetic modification is present in (a) the coding region; (b) a non-coding region; (c) a regulatory sequence; (d) an untranslated region; or (e) any combination of (a)-(d) of the genomic locus that encodes the polypeptide. In certain embodiments, the plant and/or seed is from a monocot plant. In certain embodiments, the plant is a monocot plant. In certain embodiments, the monocot plant is maize.

Provided are methods for increasing yield in a plant by expressing in a regenerable plant cell a recombinant DNA construct comprising a regulatory element operably linked to a polynucleotide encoding a polypeptide comprising an amino acid sequence that is at least 80% to about 100% identical to an amino acid sequence selected from the group consisting of SEQ ID NOS: 1-11, 23-31, 40-299, 563, 565, and 567-573; and generating the plant, wherein the plant comprises in its genome the recombinant DNA construct. In certain embodiments, the regulatory element is a heterologous promoter. In certain embodiments, the plant is a monocot plant. In certain embodiments, the monocot plant is maize. In certain embodiments, the yield is grain yield.

Further provided are methods for increasing yield in a plant by introducing in a regenerable plant cell a targeted genetic modification at a genomic locus that encodes a polypeptide comprising an amino acid sequence that is at least 80% identical to an amino acid sequence selected from the group consisting of SEQ ID NOS: 1-11, 23-31, 40-299, 563, 565, and 567-573; and generating the plant, wherein the level and/or activity of the encoded polypeptide is increased in the plant. In certain embodiments, the genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), an engineered site-specific meganucleases, or an Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes the polypeptide. In certain embodiments, the plant cell is from a monocot plant. In certain embodiments, the monocot plant is maize. In certain embodiments, the yield is grain yield.

Provided are methods for increasing photosynthetic activity in a plant by expressing in a regenerable plant cell a recombinant DNA construct comprising a regulatory element operably linked to a polynucleotide encoding a polypeptide comprising an amino acid sequence that is at least 80% identical to an amino acid sequence selected from the group consisting of SEQ ID NOS: 1-11, 23-31, 40-299, 563, 565, and 567-573; and generating the plant, wherein the plant comprises in its genome the recombinant DNA construct. In certain embodiments, the regulatory element is a heterologous promoter. In certain embodiments, the plant is a monocot plant. In certain embodiments, the monocot plant is maize.

Also provided are methods for increasing photosynthetic activity in a plant by introducing in a regenerable plant cell a targeted genetic modification at a genomic locus that encodes a polypeptide comprising an amino acid sequence that is at least 80% identical to an amino acid sequence selected from the group consisting of SEQ ID NOS: 1-11, 23-31, 40-299, 563, 565, and 567-573; and generating the plant, wherein the level and/or activity of the encoded polypeptide is increased in the plant. In certain embodiments, the genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), an engineered site-specific meganucleases, or an Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes the polypeptide. In certain embodiments, the plant cell is from a monocot plant. In certain embodiments, the monocot plant is maize.

### BRIEF DESCRIPTION OF THE DRAWINGS AND SEQUENCE LISTING

The disclosure can be more fully understood from the following detailed description and the accompanying drawings and Sequence Listing, which form a part of this application.

The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

TABLE 1A
Sequence Listing Description- Interactors and Direct Targets

| | | Functional Annotation |
|---|---|---|
| Interactors | | |
| SEQ ID NO: 1 | GRMZM2G160687 (zag2 - Zea AGAMOUS | DNA-binding transcription factor |
| | homolog2) | activity |
| SEQ ID NO: 2 | GRMZM2G160565 (bde1 - bearded-ear1; | DNA-binding transcription factor |
| | ZmMADS56) | activity |
| SEQ ID NO: 3 | GRMZM2G072582 (mads3 - MADS3; | DNA-binding transcription factor |
| | ZmZAPL) | activity |
| SEQ ID NO: 4 | GRMZM2G099522 (zmm14 - Zea mays | DNA-binding transcription factor |
| | MADS14; ZmM5) | activity |
| SEQ ID NO: 5 | GRMZM2G159397 (zmm6 - Zea mays | DNA-binding transcription factor |
| | MADS6) | activity |
| SEQ ID NO: 6 | GRMZM2G097059 (zmm7 - Zea mays | DNA-binding transcription factor |
| | MADS7) | activity |
| SEQ ID NO: 7 | GRMZM2G087095 (zmm24 - Zea mays | DNA-binding transcription factor |
| | MADS24) | activity |
| SEQ ID NO: 8 | GRMZM5G814279 (mads74 - MADS- | DNA-binding transcription factor |
| | transcription factor 74, ZmMADS47-like) | activity |
| SEQ ID NO: 9 | GRMZM2G059102 (mads68 - MADS- | DNA-binding transcription factor |
| | transcription factor 68; ZmM47) | activity |
| SEQ ID NO: 10 | GRMZM2G170365 (ZmSF2 - Zea mays | mRNA splicing |
| | splicing factor 2) | |
| SEQ ID NO: 11 | GRMZM2G086779 (ZmSFT-like - Zea mays | mRNA splicing |
| | mRNA splicing factor thioredoxin-like U5 | |
| | snRNP) | |
| SEQ ID NO: 12 | GRMZM2G160687 genomic (zag2 - Zea | DNA-binding transcription factor |
| | AGAMOUS homolog2) | activity |
| SEQ ID NO: 13 | GRMZM2G160565 genomic (bde1 - | DNA-binding transcription factor |
| | bearded-ear1; ZmMADS56) | activity |
| SEQ ID NO: 14 | GRMZM2G072582 genomic (mads3 - | DNA-binding transcription factor |
| | MADS3; ZmZAPL) | activity |
| SEQ ID NO: 15 | GRMZM2G099522 genomic (zmm14 - Zea | DNA-binding transcription factor |
| | mays MADS14; ZmM5) | activity |
| SEQ ID NO: 16 | GRMZM2G159397 genomic (zmm6 - Zea | DNA-binding transcription factor |
| | mays MADS6) | activity |
| SEQ ID NO: 17 | GRMZM2G097059 genomic (zmm7 - Zea | DNA-binding transcription factor |
| | mays MADS7) | activity |
| SEQ ID NO: 18 | GRMZM2G087095 genomic (zmm24 - Zea | DNA-binding transcription factor |
| | mays MADS24) | activity |
| SEQ ID NO: 19 | GRMZM5G814279 genomic (mads74 - | DNA-binding transcription factor |
| | MADS-transcription factor 74, | activity |
| | ZmMADS47-like) | |
| SEQ ID NO: 20 | GRMZM2G059102 genomic (mads68 - | DNA-binding transcription factor |
| | MADS-transcription factor 68; ZmM47) | activity |
| SEQ ID NO: 21 | GRMZM2G170365 genomic (ZmSF2 - Zea | mRNA splicing |
| | mays splicing factor 2) | |
| SEQ ID NO: 22 | GRMZM2G086779 genomic (ZmSFT-like - | mRNA splicing |
| | Zea mays mRNA splicing factor | |
| | thioredoxin-like U5 snRNP) | |
| Direct Targets | | |
| SEQ ID NO: 23 | GRMZM2G036880 (light-harvesting | integral component of thylakoid |
| | complex I chlorophyll a/b binding protein | membrane |
| | 1 (LHCA1) like) | |
| SEQ ID NO: 24 | GRMZM2G117412 (Chlorophyll a-b | photosynthesis |
| | binding protein) | |
| SEQ ID NO: 25 | GRMZM2G176840 (PSBP-like protein 1, | photosynthesis |
| | chloroplastic) | |
| SEQ ID NO: 26 | GRMZM2G149428 (Ihcb5 - light | photosynthesis |
| | harvesting chlorophyll a/b binding | |
| | protein5) | |
| SEQ ID NO: 27 | GRMZM2G112728 (all-trans-nonaprenyl- | photosynthesis |
| | diphosphate synthase) | |
| SEQ ID NO: 28 | GRMZM2G306345 (pdk1 - pyruvate, | photosynthesis |
| | orthophosphate dikinase1) | |
| SEQ ID NO: 29 | GRMZM2G306345 (pdk1 - pyruvate, | photosynthesis |
| | orthophosphate dikinase1) | |
| SEQ ID NO: 30 | GRMZM2G459166 (F-box protein GID2 | giberellin signaling |
| | (GID2, SLY1)) | |
| SEQ ID NO: 31 | GRMZM2G073427 (bzip111 - bZIP- | DNA-binding transcription factor |
| | transcription factor 111) | activity |
| SEQ ID NO: 32 | GRMZM2G036880 genomic (light- | integral component of thylakoid |
| | harvesting complex I chlorophyll a/b | membrane |
| | binding protein 1 (LHCA1) like) | |
| SEQ ID NO: 33 | GRMZM2G117412 genomic (Chlorophyll | photosynthesis |
| | a-b binding protein) | |
| SEQ ID NO: 34 | GRMZM2G176840 genomic (PSBP-like | photosynthesis |
| | protein 1, chloroplastic) | |
| SEQ ID NO: 35 | GRMZM2G149428 genomic (Ihcb5 - light | photosynthesis |
| | harvesting chlorophyll a/b binding | |
| | protein5) | |
| SEQ ID NO: 36 | GRMZM2G112728 genomic (all-trans- | photosynthesis |
| | nonaprenyl-diphosphate synthase) | |
| SEQ ID NO: 37 | GRMZM2G306345 genomic (pdk1 - | photosynthesis |
| | pyruvate, orthophosphate dikinase1) | |
| SEQ ID NO: 38 | GRMZM2G459166 genomic (F-box | giberellin signaling |
| | protein GID2 (GID2, SLY1)) | |
| SEQ ID NO: 39 | GRMZM2G073427 genomic (bzip111 - | DNA-binding transcription factor |
| | bZIP-transcription factor 111) | activity |

TABLE 1B
Sequence Listing Description- Differentially Expressed Genes

| Differentially Expressed Genes | | Functional Annotation |
|---|---|---|
| SEQ ID NO: 40 | Zm00001d022088 Protein Agamous-like | DNA-binding transcription factor |
| | MADS-box protein AGL8 | activity |
| SEQ ID NO: 41 | Zm00001d023455 Protein Transcription | DNA-binding transcription factor |
| | repressor OFP13 | activity |
| SEQ ID NO: 42 | Zm00001d023456 Protein Transcription | DNA-binding transcription factor |
| | repressor OFP6 | activity |
| SEQ ID NO: 43 | Zm00001d038273 Protein Coronatine- | shade avoidance |
| | insensitive protein 1 | |
| SEQ ID NO: 44 | Zm00001d004053 Protein hypothetical | None identified |
| | protein | |
| SEQ ID NO: 45 | Zm00001d029183 Protein cytochrome | isoflavone 2′-hydroxylase activity |
| | P450 family 81 subfamily D | |
| | polypeptide 8 | |
| SEQ ID NO: 46 | Zm00001d051194 Protein Arginine | arginine decarboxylase activity |
| | decarboxylase | |
| SEQ ID NO: 47 | Zm00001d033132 Protein photosystem II | photosynthesis |
| | light harvesting complex gene 2.1 | |
| SEQ ID NO: 48 | Zm00001d029215 Protein Villin-2 | actin filament bundle assembly |
| SEQ ID NO: 49 | Zm00001d033543 Protein Integral | cellular carbohydrate metabolic |
| | membrane HPP family protein | process |
| SEQ ID NO: 50 | Zm00001d053925 Protein hypothetical | chlorophyll biosynthetic process |
| | protein | |
| SEQ ID NO: 51 | Zm00001d028269 Protein hypothetical | DNA-binding transcription factor |
| | protein | activity |
| SEQ ID NO: 52 | Zm00001d033544 Protein | transferase activity, transferring |
| | Glycosyltransferase family protein 64 | hexosyl groups |
| | protein C5 | |
| SEQ ID NO: 53 | Zm00001d034015 Protein exoglucanasel | glucan exo-1,3-beta-glucosidase |
| | | activity |
| SEQ ID NO: 54 | Zm00001d027743 Protein | sugar mediated signaling pathway |
| | Serine/threonine-protein kinase EDR1 | |
| SEQ ID NO: 55 | Zm00001d048311 Protein Sucrose | sucrose transmembrane transporter |
| | transport protein SUC3 | activity |
| SEQ ID NO: 56 | Zm00001d047256 Protein Protein kinase | signal transduction |
| | domain superfamily protein | |
| SEQ ID NO: 57 | Zm00001d048720 Protein Glutaredoxin- | electron transport chain |
| | C13 | |
| SEQ ID NO: 58 | Zm00001d023426 Protein hypothetical | chlorophyll biosynthetic process |
| | protein | |
| SEQ ID NO: 59 | Zm00001d004331 Protein hypothetical | regulation of transcription, DNA- |
| | protein | templated |
| SEQ ID NO: 60 | Zm00001d050748 Protein proline-rich | ATPase activity, coupled to |
| | family protein | transmembrane movement of |
| | | substances |
| SEQ ID NO: 61 | Zm00001d010321 Protein pyruvate | pyruvate, phosphate dikinase |
| | orthophosphate dikinase2 | activity |
| SEQ ID NO: 62 | Zm00001d004894 Protein ribulose | ribulose-bisphosphate carboxylase |
| | bisphosphate carboxylase small subunit2 | activity |
| SEQ ID NO: 63 | Zm00001d042346 Protein alpha/beta- | haloalkane dehalogenase activity |
| | Hydrolases superfamily protein | |
| SEQ ID NO: 64 | Zm00001d031657 Protein putative | acid phosphatase activity |
| | inactive purple acid phosphatase 27 | |
| SEQ ID NO: 65 | Zm00001d008178 Protein ABC | basipetal auxin transport |
| | transporter B family member 21 | |
| SEQ ID NO: 66 | Zm00001d043044 Protein Sec1/munc18- | kinase activity |
| | like (SM) proteins superfamily | |
| SEQ ID NO: 67 | Zm00001d018623 Protein Photosynthetic | electron transporter, transferring |
| | NDH subunit of lumenal location 3 | electrons within the cyclic electron |
| | chloroplastic | transport pathway of |
| | | photosynthesis activity |
| SEQ ID NO: 68 | Zm00001d043095 Protein Zinc finger | chloroplast organization |
| | protein VAR3 chloroplastic | |
| SEQ ID NO: 69 | Zm00001d029062 Protein Vicilin-like | nutrient reservoir activity |
| | seed storage protein | |
| SEQ ID NO: 70 | Zm00001d011900 Protein RNA-binding | RNA binding |
| | protein BRN1 | |
| SEQ ID NO: 71 | Zm00001d010672 Protein Metacaspase | phosphoglycerate kinase activity |
| | type II | |
| SEQ ID NO: 72 | Zm00001d011183 Protein thiamine | thiazole biosynthetic process |
| | biosynthesis1 | |
| SEQ ID NO: 73 | Zm00001d037103 Protein Peroxiredoxin | peroxiredoxin activity |
| | Q chloroplastic | |
| SEQ ID NO: 74 | Zm00001d009028 Protein Triose | transporter activity |
| | phosphate/phosphate translocator, | |
| | chloroplastic | |
| SEQ ID NO: 75 | Zm00001d013937 Protein | chlorophyll biosynthetic process |
| | Protochlorophyllide reductase C | |
| | chloroplastic | |
| SEQ ID NO: 76 | Zm00001d002873 Protein UPF0426 | plastoglobule |
| | protein chloroplastic | |
| SEQ ID NO: 77 | Zm00001d037362 Protein DNA | DNA topoisomerase activity |
| | topoisomerase type IA core | |
| SEQ ID NO: 78 | Zm00001d026404 Protein hypothetical | arginine catabolic process to |
| | protein | glutamate |
| SEQ ID NO: 79 | Zm00001d047255 Protein 3-oxoacyl- | 3-oxoacyl-[acyl-carrier-protein] |
| | [acyl-carrier-protein] synthase II | synthase activity |
| | chloroplastic | |
| SEQ ID NO: 80 | Zm00001d031253 Protein dicarboxylic | transporter activity |
| | acid transporter2 | |
| SEQ ID NO: 81 | Zm00001d027576 Protein hypothetical | voltage-gated anion channel |
| | protein | activity |
| SEQ ID NO: 82 | Zm00001d052595 Protein Ribulose | ribulose-bisphosphate carboxylase |
| | bisphosphate carboxylase small chain, | activity |
| | chloroplastic | |
| SEQ ID NO: 83 | Zm00001d013367 Protein Tubulin alpha- | structural molecule activity |
| | 4 chain | |
| SEQ ID NO: 84 | Zm00001d046001 Protein Triose | transmembrane transporter activity |
| | phosphate/phosphate translocator TPT | |
| | chloroplastic | |
| SEQ ID NO: 85 | Zm00001d008963 Protein GDT1-like | chloroplast membrane |
| | protein 1 chloroplastic | |
| SEQ ID NO: 86 | Zm00001d048515 Protein Stress | gene silencing |
| | responsive alpha-beta barrel domain | |
| | protein | |
| SEQ ID NO: 87 | Zm00001d052595 Protein Ribulose | ribulose-bisphosphate carboxylase |
| | bisphosphate carboxylase small chain, | activity |
| | chloroplastic | |
| SEQ ID NO: 88 | Zm00001d042049 Protein Ferredoxin | photosynthetic electron transport |
| | | in photosystem I |
| SEQ ID NO: 89 | Zm00001d040242 Protein Nuclear | cellular macromolecule metabolic |
| | transport factor 2 (NTF2) family protein | process |
| SEQ ID NO: 90 | Zm00001d042697 Protein photosystem II | PSII associated light-harvesting |
| | subunit PsbS1 | complex II |
| SEQ ID NO: 91 | Zm00001d019518 Protein Photosystem I | photosystem I reaction center |
| | reaction center subunit IV A | |
| SEQ ID NO: 92 | Zm00001d048313 Protein NAD(P)-linked | photosystem II assembly |
| | oxidoreductase superfamily protein | |
| SEQ ID NO: 93 | Zm00001d033150 Protein hypothetical | transcription regulatory region |
| | protein | sequence-specific DNA binding |
| SEQ ID NO: 94 | Zm00001d007858 Protein Pyridoxal | oxidoreductase activity |
| | reductase chloroplastic | |
| SEQ ID NO: 95 | Zm00001d003588 Protein Ras-related | protein binding |
| | protein RABD1 | |
| SEQ ID NO: 96 | Zm00001d036903 Protein Plant Tudor- | RNA binding |
| | like RNA-binding protein | |
| SEQ ID NO: 97 | Zm00001d031484 Protein PsbP domain- | photosystem II oxygen evolving |
| | containing protein 3 chloroplastic | complex |
| SEQ ID NO: 98 | Zm00001d018157 Protein light harvesting | photosynthesis, light harvesting in |
| | complex a/b protein4 | photosystem I |
| SEQ ID NO: 99 | Zm00001d005814 Protein Photosystem I | photosynthesis, light harvesting in |
| | chlorophyll a/b-binding protein 6 | photosystem I |
| | chloroplastic | |
| SEQ ID NO: 100 | Zm00001d033338 Protein NAD(P)-linked | oxidoreductase activity |
| | oxidoreductase superfamily protein | |
| SEQ ID NO: 101 | Zm00001d053446 Protein potassium | voltage-gated potassium channel |
| | channel3 | activity |
| SEQ ID NO: 102 | Zm00001d026603 Protein Magnesium- | photosynthesis, light reaction |
| | chelatase subunit ChlH chloroplastic | |
| SEQ ID NO: 103 | Zm00001d027841 Protein Ribulose- | pentose-phosphate shunt, non- |
| | phosphate 3-epimerase | oxidative branch |
| SEQ ID NO: 104 | Zm00001d030048 Protein pfkB-like | isopentenyl diphosphate |
| | carbohydrate kinase family protein | biosynthetic process, |
| | | methylerythritol 4-phosphate |
| | | pathway |
| SEQ ID NO: 105 | Zm00001d005346 Protein Aldo-keto | oxidoreductase activity |
| | reductase/oxidoreductase | |
| SEQ ID NO: 106 | Zm00001d023706 Protein thioredoxin M1 | transcription coregulator activity |
| SEQ ID NO: 107 | Zm00001d042050 Protein Protein | chlorophyll biosynthetic process |
| | RETICULATA-RELATED 4 | |
| | chloroplastic | |
| SEQ ID NO: 108 | Zm00001d042533 Protein Trigger factor | protein transport |
| SEQ ID NO: 109 | Zm00001d022590 Protein hypothetical | transcription, DNA-templated |
| | protein | |
| SEQ ID NO: 110 | Zm00001d045431 Protein Enolase 1 | phosphopyruvate hydratase |
| | | activity |
| SEQ ID NO: 111 | Zm00001d047743 Protein fatty acid | oxidoreductase activity, acting on |
| | desaturase7 | paired donors, with oxidation of a |
| | | pair of donors resulting in the |
| | | reduction of molecular oxygen to |
| | | two molecules of water |
| SEQ ID NO: 112 | Zm00001d036738 Protein S-adenosyl-L- | methyltransferase activity |
| | methionine-dependent methyltransferase | |
| | superfamily protein | |
| SEQ ID NO: 113 | Zm00001d018274 Protein Isoleucine-- | valyl-tRNA aminoacylation |
| | tRNA ligase chloroplastic/mitochondrial | |
| SEQ ID NO: 114 | Zm00001d035003 Protein ferredoxin2 | electron transfer activity |
| SEQ ID NO: 115 | Zm00001d027321 Protein hypothetical | peptidyl-prolyl cis-trans isomerase |
| | protein | activity |
| SEQ ID NO: 116 | Zm00001d014284 Protein CMV 1a | methyltransferase activity |
| | interacting protein 1 | |
| SEQ ID NO: 117 | Zm00001d036340 Protein photosystem | photosystem II |
| | II1 | |
| SEQ ID NO: 118 | Zm00001d003767 Protein Photosystem I | photosynthesis, light harvesting in |
| | subunit O | photosystem I |
| SEQ ID NO: 119 | Zm00001d031997 Protein NAD(P)- | nucleotide binding |
| | binding Rossmann-fold superfamily | |
| | protein | |
| SEQ ID NO: 120 | Zm00001d019479 Protein granule-bound | NDP-glucose-starch |
| | starch synthase 1b | glucosyltransferase activity |
| SEQ ID NO: 121 | Zm00001d024148 Protein Photosynthetic | photosynthetic electron transport |
| | NDH subunit of subcomplex B 1 | in photosystem I |
| | chloroplastic | |
| SEQ ID NO: 122 | Zm00001d038579 Protein | photosynthesis, light reaction |
| | Phosphoglycerate kinase | |
| SEQ ID NO: 123 | Zm00001d037273 Protein Peptide | electron transport chain |
| | methionine sulfoxide reductase msrB | |
| SEQ ID NO: 124 | Zm00001d025845 Protein hypothetical | chloroplast thylakoid membrane |
| | protein | |
| SEQ ID NO: 125 | Zm00001d027422 Protein PsbP-like | chloroplast photosystem II |
| | protein 1 chloroplastic | |
| SEQ ID NO: 126 | Zm00001d024519 Protein rubredoxin | iron ion binding |
| | family protein | |
| SEQ ID NO: 127 | Zm00001d040163 Protein deoxy xylulose | 1-deoxy-D-xylulose-5-phosphate |
| | reductoisomerase1 | reductoisomerase activity |
| SEQ ID NO: 128 | Zm00001d012868 Protein carotene | oxidoreductase activity |
| | isomerase3 | |
| SEQ ID NO: 129 | Zm00001d021763 Protein photosystem II | photosynthesis, light harvesting in |
| | subunit29 | photosystem I |
| SEQ ID NO: 130 | Zm00001d035761 Protein Peptidyl-prolyl | peptidyl-prolyl cis-trans isomerase |
| | cis-trans isomerase | activity |
| SEQ ID NO: 131 | Zm00001d032301 Protein S-adenosyl-L- | methyltransferase activity |
| | methionine-dependent methyltransferase | |
| | superfamily protein | |
| SEQ ID NO: 132 | Zm00001d028562 Protein Fructose-1,6- | photosynthetic electron transport |
| | bisphosphatase | in photosystem I |
| SEQ ID NO: 133 | Zm00001d034538 Protein Rubredoxin- | iron ion binding |
| | like superfamily protein | |
| SEQ ID NO: 134 | Zm00001d048116 Protein hypothetical | isopentenyl diphosphate |
| | protein | biosynthetic process, |
| | | methylerythritol 4-phosphate |
| | | pathway |
| SEQ ID NO: 135 | Zm00001d048998 Protein Chlorophyll a- | photosynthesis |
| | b binding protein CP26 chloroplastic | |
| SEQ ID NO: 136 | Zm00001d049490 Protein Protein | translation initiation factor activity |
| | CHLORORESPIRATORY REDUCTION | |
| | 6 chloroplastic | |
| SEQ ID NO: 137 | Zm00001d015975 Protein putative | lactoylglutathione lyase activity |
| | lactoylglutathione lyase chloroplastic | |
| SEQ ID NO: 138 | Zm00001d015613 Protein Protein TIC 21 | protein import into chloroplast |
| | chloroplastic | stroma |
| SEQ ID NO: 139 | Zm00001d039258 Protein Triose | glucose-6-phosphate |
| | phosphate/phosphate translocator TPT | transmembrane transporter activity |
| | chloroplastic | |
| SEQ ID NO: 140 | Zm00001d007267 Protein light harvesting | photosynthesis, light harvesting in |
| | chlorophyll a/b binding protein5 | photosystem I |
| SEQ ID NO: 141 | Zm00001d033383 Protein | thiamine biosynthetic process |
| | hydroxymethylpyrimidine phosphate | |
| | synthase1 | |
| SEQ ID NO: 142 | Zm00001d026645 Protein K(+) efflux | solute: proton antiporter activity |
| | antiporter 2 chloroplastic | |
| SEQ ID NO: 143 | Zm00001d023757 Protein NAD(P)H- | heat shock protein binding |
| | quinone oxidoreductase subunit U | |
| | chloroplastic | |
| SEQ ID NO: 144 | Zm00001d034005 Protein evolutionarily | oxidation-reduction process |
| | conserved C-terminal region 2 | |
| SEQ ID NO: 145 | Zm00001d022381 Protein NADPH- | hydrogen peroxide catabolic |
| | dependent thioredoxin reductase 3 | process |
| SEQ ID NO: 146 | Zm00001d031962 Protein hypothetical | protein dephosphorylation |
| | protein | |
| SEQ ID NO: 147 | Zm00001d005446 Protein Photosystem I | photosystem I reaction center |
| | reaction center subunit IV A | |
| SEQ ID NO: 148 | Zm00001d027511 Protein Catalase | hydrogen peroxide catabolic |
| | isozyme 2 | process |
| SEQ ID NO: 149 | Zm00001d017178 Protein hypothetical | primary metabolic process |
| | protein | |
| SEQ ID NO: 150 | Zm00001d038947 Protein putative | electron transfer activity |
| | galacturonosyltransferase-like 9 | |
| SEQ ID NO: 151 | Zm00001d034739 Protein | acetylpyruvate hydrolase activity |
| | Fumarylacetoacetate (FAA) hydrolase | |
| | family | |
| SEQ ID NO: 152 | Zm00001d044745 Protein Alanine--tRNA | alanyl-tRNA aminoacylation |
| | ligase chloroplastic/mitochondrial | |
| SEQ ID NO: 153 | Zm00001d038491 Protein | N-acetyltransferase activity |
| | Acetyltransferase NSI | |
| SEQ ID NO: 154 | Zm00001d014445 Protein Protein kinase | kinase activity |
| | superfamily protein | |
| SEQ ID NO: 155 | Zm00001d039745 Protein Protein | phosphoprotein phosphatase |
| | phosphatase 2C | activity |
| SEQ ID NO: 156 | Zm00001d038485 Protein | photosystem II stabilization |
| | Serine/threonine-protein kinase STN8 | |
| | chloroplastic | |
| SEQ ID NO: 157 | Zm00001d012287 Protein hypothetical | starch biosynthetic process |
| | protein | |
| SEQ ID NO: 158 | Zm00001d027694 Protein Solanesyl | plastoquinone biosynthetic process |
| | diphosphate synthase 2 chloroplastic | |
| SEQ ID NO: 159 | Zm00001d003470 Protein putative | response to abscisic acid |
| | plastid-lipid-associated protein 2 | |
| | chloroplastic | |
| SEQ ID NO: 160 | Zm00001d019180 Protein Nudix | hydrolase activity |
| | hydrolase 16 mitochondrial | |
| SEQ ID NO: 161 | Zm00001d007394 Protein Rubredoxin- | iron ion binding |
| | like superfamily protein | |
| SEQ ID NO: 162 | Zm00001d053432 Protein iron-sulfur | electron transporter, transferring |
| | protein2 | electrons within cytochrome b6/f |
| | | complex of photosystem II activity |
| SEQ ID NO: 163 | Zm00001d039900 Protein putative zinc | metalloendopeptidase activity |
| | metalloprotease EGY2 chloroplastic | |
| SEQ ID NO: 164 | Zm00001d050810 Protein 2-hydroxy-3- | pentose-phosphate shunt |
| | oxopropionate reductase | |
| SEQ ID NO: 165 | Zm00001d016802 Protein L-ascorbate | L-ascorbate peroxidase activity |
| | peroxidase S chloroplastic/mitochondrial | |
| SEQ ID NO: 166 | Zm00001d004978 Protein Saccharopine | oxidoreductase activity |
| | dehydrogenase | |
| SEQ ID NO: 167 | Zm00001d028924 Protein NifU-like | iron-sulfur cluster assembly |
| | protein 1 chloroplastic | |
| SEQ ID NO: 168 | Zm00001d002815 Protein NAD(P)H- | oxidoreductase activity, acting on |
| | quinone oxidoreductase subunit M | NAD(P)H, quinone or similar |
| | chloroplastic | compound as acceptor |
| SEQ ID NO: 169 | Zm00001d029065 Protein Protein LRP16 | regulation of transcription, DNA- |
| | | templated |
| SEQ ID NO: 170 | Zm00001d052184 Protein Protein | chloroplast envelope |
| | CURVATURE THYLAKOID ID | |
| | chloroplastic | |
| SEQ ID NO: 171 | Zm00001d038337 Protein DAR GTPase 3 | GTP binding |
| | chloroplastic | |
| SEQ ID NO: 172 | Zm00001d003713 Protein Exocyst | pollen tube growth |
| | complex component SEC5A | |
| SEQ ID NO: 173 | Zm00001d031953 Protein Thioredoxin | protein disulfide oxidoreductase |
| | family protein | activity |
| SEQ ID NO: 174 | Zm00001d053576 Protein putative | eukaryotic translation elongation |
| | elongation factor 1-gamma 2 | factor 1 complex |
| SEQ ID NO: 175 | Zm00001d000123 Protein NAD(P)- | response to oxidative stress |
| | binding Rossmann-fold superfamily | |
| | protein | |
| SEQ ID NO: 176 | Zm00001d045431 Protein Enolase 1 | phosphopyruvate hydratase |
| | | activity |
| SEQ ID NO: 177 | Zm00001d036630 Protein Filamentation | metalloendopeptidase activity |
| | temperature-sensitive H 2B | |
| SEQ ID NO: 178 | Zm00001d048515 Protein Stress | gene silencing |
| | responsive alpha-beta barrel domain | |
| | protein | |
| SEQ ID NO: 179 | Zm00001d021310 Protein | reductive pentose-phosphate cycle |
| | Triosephosphate isomerase | |
| SEQ ID NO: 180 | Zm00001d042353 Protein sucrose | sucrose synthase activity |
| | phosphate synthase2 | |
| SEQ ID NO: 181 | Zm00001d050150 Protein Adenylate | adenylate kinase activity |
| | kinase 5 chloroplastic | |
| SEQ ID NO: 182 | Zm00001d025545 Protein zeaxanthin | abscisic acid biosynthetic process |
| | epoxidase2 | |
| SEQ ID NO: 183 | Zm00001d012168 Protein Membrane- | chloroplast thylakoid membrane |
| | associated protein VIPP1 chloroplastic | |
| SEQ ID NO: 184 | Zm00001d011581 Protein Peroxiredoxin- | peroxiredoxin activity |
| | 2B | |
| SEQ ID NO: 185 | Zm00001d018030 Protein Photosynthetic | regulation of transcription by RNA |
| | NDH subunit of subcomplex B 4 | polymerase II |
| | chloroplastic | |
| SEQ ID NO: 186 | Zm00001d018145 Protein Presequence | protein processing |
| | protease 2 chloroplastic/mitochondrial | |
| SEQ ID NO: 187 | Zm00001d040221 Protein Peptidase | metalloendopeptidase activity |
| | family M48 family protein | |
| SEQ ID NO: 188 | Zm00001d033594 Protein Myelin- | phosphorus metabolic process |
| | associated oligodendrocyte basic protein | |
| | isoform 1 | |
| SEQ ID NO: 189 | Zm00001d008625 Protein Inner | thylakoid membrane organization |
| | membrane protein ALBINO3 | |
| | chloroplastic | |
| SEQ ID NO: 190 | Zm00001d032380 Protein acclimation of | photosystem II assembly |
| | photosynthesis to environment | |
| SEQ ID NO: 191 | Zm00001d045575 Protein Ferredoxin- | photosynthetic electron transport |
| | NADP reductase leaf isozyme 1 | in photosystem I |
| | chloroplastic | |
| SEQ ID NO: 192 | Zm00001d042526 Protein | regulation of stomatai closure |
| | Rhodanese/Cell cycle control phosphatase | |
| | superfamily protein | |
| SEQ ID NO: 193 | Zm00001d043168 Protein MAR-binding | photosystem II assembly |
| | filament-like protein 1-1 isoform 2 | |
| SEQ ID NO: 194 | Zm00001d053545 Protein NAD(P)- | nucleotide binding |
| | binding Rossmann-fold superfamily | |
| | protein | |
| SEQ ID NO: 195 | Zm00001d053981 Protein putative | pyridoxal phosphate biosynthetic |
| | pyridoxal 5′-phosphate synthase subunit | process |
| | PDX2 | |
| SEQ ID NO: 196 | Zm00001d017746 Protein vitamin E | tocopherol O-methyltransferase |
| | synthesis4 | activity |
| SEQ ID NO: 197 | Zm00001d012083 Protein Thioredoxin F- | pentose-phosphate shunt |
| | type chloroplastic | |
| SEQ ID NO: 198 | Zm00001d024718 Protein Beta-carotene | strigolactone biosynthetic process |
| | isomerase D27 chloroplastic | |
| SEQ ID NO: 199 | Zm00001d021621 Protein | 3′-5′ exonuclease activity |
| | Polynucleotidyl transferase ribonuclease | |
| | H fold protein with HRDC domain | |
| SEQ ID NO: 200 | Zm00001d015004 Protein Protein LOW | aromatic amino acid family |
| | PSII ACCUMULATION 3 chloroplastic | biosynthetic process |
| SEQ ID NO: 201 | Zm00001d039131 Protein ADP glucose | glucose-1-phosphate |
| | pyrophosphorylase2 | adenylyltransferase activity |
| SEQ ID NO: 202 | Zm00001d044970 Protein putative | protein tyrosine phosphatase |
| | tyrosine-protein phosphatase | activity |
| SEQ ID NO: 203 | Zm00001d018401 Protein plastid | positive regulation of ubiquitin |
| | transcriptionally active 17 | protein ligase activity |
| SEQ ID NO: 204 | Zm00001d015975 Protein putative | lactoylglutathione lyase activity |
| | lactoylglutathione lyase chloroplastic | |
| SEQ ID NO: 205 | Zm00001d013428 Protein | phosphoglucomutase activity |
| | phosphoglucomutase2 | |
| SEQ ID NO: 206 | Zm00001d021246 Protein Ras-related | chloroplast thylakoid membrane |
| | protein RABA3 | |
| SEQ ID NO: 207 | Zm00001d007921 Protein Tic62 protein | oxidation-reduction process |
| SEQ ID NO: 208 | Zm00001d027511 Protein Catalase | hydrogen peroxide catabolic |
| | isozyme 2 | process |
| SEQ ID NO: 209 | Zm00001d027309 Protein Phosphoglucan | intracellular signal transduction |
| | phosphatase DSP4 chloroplastic | |
| SEQ ID NO: 210 | Zm00001d025544 Protein Zeaxanthin | zeaxanthin epoxidase [overall] |
| | epoxidase chloroplastic | activity |
| SEQ ID NO: 211 | Zm00001d039276 Protein Ubiquitin | cellular metabolic process |
| | ligase SINAT3 | |
| SEQ ID NO: 212 | Zm00001d003512 Protein Zeaxanthin | zeaxanthin epoxidase [overall] |
| | epoxidase chloroplastic | activity |
| SEQ ID NO: 213 | Zm00001d053861 Protein Putative | GTP binding |
| | translation elongation factor family | |
| | protein | |
| SEQ ID NO: 214 | Zm00001d038894 Protein | circadian rhythm |
| | Serine/threonine-protein kinase STN7 | |
| | chloroplastic | |
| SEQ ID NO: 215 | Zm00001d051321 Protein ATP- | metalloendopeptidase activity |
| | dependent zinc metalloprotease FTSH 7 | |
| | chloroplastic | |
| SEQ ID NO: 216 | Zm00001d016826 Protein proline-rich | anatomical structure |
| | family protein | morphogenesis |
| SEQ ID NO: 217 | Zm00001d016854 Protein Ferrochelatase | starch biosynthetic process |
| SEQ ID NO: 218 | Zm00001d017435 Protein hypothetical | none |
| | protein | |
| SEQ ID NO: 219 | Zm00001d048373 Protein Carotenoid | oxidoreductase activity, acting on |
| | 910(9′10′)-cleavage dioxygenase 1 | single donors with incorporation of |
| | | molecular oxygen, incorporation |
| | | of two atoms of oxygen |
| SEQ ID NO: 220 | Zm00001d018901 Protein protein | protein binding |
| | containing PDZ domain a K-box domain | |
| | and a TPR region | |
| SEQ ID NO: 221 | Zm00001d019454 Protein PGR5-like | photosynthetic electron transport |
| | protein 1B chloroplastic | in photosystem I |
| SEQ ID NO: 222 | Zm00001d031899 Protein malate | malate dehydrogenase (NADP+) |
| | dehydrogenase6 | activity |
| SEQ ID NO: 223 | Zm00001d045706 Protein Restorer of | aldehyde dehydrogenase (NAD) |
| | fertility2 | activity |
| SEQ ID NO: 224 | Zm00001d035869 Protein Protein MEI2- | nucleotide binding |
| | like 5 | |
| SEQ ID NO: 225 | Zm00001d053262 Protein calcium- | protein localization |
| | dependent lipid-binding family protein | |
| SEQ ID NO: 226 | Zm00001d017958 Protein Glutamine | glutamine biosynthetic process |
| | synthetase root isozyme 3 | |
| SEQ ID NO: 227 | Zm00001d007113 Protein Xylose | xylose isomerase activity |
| | isomerase | |
| SEQ ID NO: 228 | Zm00001d051650 Protein Sucrose | transcription, DNA-templated |
| | cleavage protein-like protein | |
| SEQ ID NO: 229 | Zm00001d015138 Protein hypothetical | nuclear pore |
| | protein | |
| SEQ ID NO: 230 | Zm00001d030103 Protein putative | xyloglucan: xyloglucosyl |
| | xyloglucan endotransglucosylase/ | transferase activity |
| | hydrolase protein 30 | |
| SEQ ID NO: 231 | Zm00001d021846 Protein Insulin- | metalloendopeptidase activity |
| | degrading enzyme-like 1 peroxisomal | |
| SEQ ID NO: 232 | Zm00001d041576 Protein Transcription | regulation of transcription, DNA- |
| | factor MYB48 | templated |
| SEQ ID NO: 233 | Zm00001d005391 Protein Cysteine | cysteine-type endopeptidase |
| | proteinases superfamily protein | activity |
| SEQ ID NO: 234 | Zm00001d009022 Protein NmrA-like | 166 nucleotide binding |
| | negative transcriptional regulator family | |
| | protein | |
| SEQ ID NO: 235 | Zm00001d041343 Protein putative | defense response |
| | disease resistance protein | |
| SEQ ID NO: 236 | Zm00001d039965 Protein Delta(7)-sterol- | fatty acid biosynthetic process |
| | C5(6)-desaturase 1 | |
| SEQ ID NO: 237 | Zm00001d029047 Protein Receptor-like | signaling receptor activity |
| | protein kinase FERONIA | |
| SEQ ID NO: 238 | Zm00001d028230 Protein Sugar transport | sucrose transport |
| | protein 13 | |
| SEQ ID NO: 239 | Zm00001d013243 Protein Cyclic | plant-type hypersensitive response |
| | nucleotide-gated ion channel 2 | |
| SEQ ID NO: 240 | Zm00001d032926 Protein | chlorophyll catabolic process |
| | chlorophyllase2 | |
| SEQ ID NO: 241 | Zm00001d045667 Protein Protein NRT1/ | nitrate assimilation |
| | PTR FAMILY 3.1 | |
| SEQ ID NO: 242 | Zm00001d021846 Protein Insulin- | metalloendopeptidase activity |
| | degrading enzyme-like 1 peroxisomal | |
| SEQ ID NO: 243 | Zm00001d033469 Protein Ferredoxin | electron transfer activity |
| SEQ ID NO: 244 | Zm00001d019563 Protein Aquaporin | transporter activity |
| | PIP2-1 | |
| SEQ ID NO: 245 | Zm00001d003866 Protein hypothetical | cell wall organization |
| | protein | |
| SEQ ID NO: 246 | Zm00001d039325 Protein Putative | signaling receptor activity |
| | inactive receptor-like protein kinase | |
| SEQ ID NO: 247 | Zm00001d023516 Protein Salt stress- | mannose binding |
| | induced protein | |
| SEQ ID NO: 248 | Zm00001d050918 Protein hypothetical | aldehyde dehydrogenase (NAD) |
| | protein | activity |
| SEQ ID NO: 249 | Zm00001d025253 Protein hypothetical | none |
| | protein | |
| SEQ ID NO: 250 | Zm00001d024499 Protein Nuclear factor | aminoacyl-tRNA hydrolase |
| | 1 A-type isoform 2 | activity |
| SEQ ID NO: 251 | Zm00001d045948 Protein Protein | drug transmembrane transporter |
| | DETOXIFICATION 16 | activity |
| SEQ ID NO: 252 | Zm00001d021569 Protein Transparent | drug transmembrane transporter |
| | testa 12 protein | activity |
| SEQ ID NO: 253 | Zm00001d053327 Protein Galactoside 2- | xyloglucan biosynthetic process |
| | alpha-L-fucosyltransferase | |
| SEQ ID NO: 254 | Zm00001d047208 Protein WAT1-related | auxin-activated signaling pathway |
| | protein | |
| SEQ ID NO: 255 | Zm00001d007687 Protein Tropinone | response to karrikin |
| | reductase-like protein | |
| SEQ ID NO: 256 | Zm00001d015126 Protein response to low | pyridoxamine-phosphate oxidase |
| | sulfur 3 | activity |
| SEQ ID NO: 257 | Zm00001d034781 Protein G-type lectin | multicellular organism |
| | S-receptor-like serine/threonine-protein | development |
| | kinase | |
| SEQ ID NO: 258 | Zm00001d016655 Protein hypothetical | voltage-gated potassium channel |
| | protein | activity |
| SEQ ID NO: 259 | Zm00001d043517 Protein Peptidase M28 | regulation of inflorescence |
| | family protein | meristem growth |
| SEQ ID NO: 260 | Zm00001d045667 Protein Protein NRT1/ | nitrate assimilation |
| | PTR FAMILY 3.1 | |
| SEQ ID NO: 261 | Zm00001d000126 Function unknown | voltage-gated potassium channel |
| | | activity |
| SEQ ID NO: 262 | Zm00001d029706 Glutathione S- | glutathione transferase activity |
| | transferase GSTU6-like protein | |
| SEQ ID NO: 263 | Zm00001d029321 Cationic amino acid | amino acid transmembrane |
| | transporter | transporter activity |
| SEQ ID NO: 264 | Zm00001d014701 Transcription regulator | regulation of transcription, DNA- |
| | HTH, Myb-type, DNA-binding protein | templated |
| SEQ ID NO: 265 | Zm00001d049624 Glutamate-rich WD | zinc ion transmembrane transport |
| | repeat-containing protein 1-like protein | |
| SEQ ID NO: 266 | Zm00001d034359 Zinc finger, C2H2- | nucleic acid binding |
| | type/integrase, DNA-binding protein | |
| SEQ ID NO: 267 | Zm00001d033924 Cell wall protein | proton-transporting ATP synthase |
| | pherophorin-C10 (PHC10) | activity, rotational mechanism |
| SEQ ID NO: 268 | Zm00001d032222 UDP- | glucuronosyltransferase activity |
| | glycosyltransferase 85A2-like protein | |
| SEQ ID NO: 269 | Zm00001d018155 Galactoside 2-alpha-L- | cell wall biogenesis |
| | fucosyltransferase-like protein | |
| SEQ ID NO: 270 | Zm00001d036263 Receptor protein | protein serine/threonine kinase |
| | serine/threonine kinase | activity |
| SEQ ID NO: 271 | Zm00001d044043 Acetylajmaline | lipid catabolic process |
| | esterase | |
| SEQ ID NO: 272 | Zm00001d027425 MADS-box | transcription, DNA-templated |
| | transcription factor 56-like protein | |
| SEQ ID NO: 273 | Zm00001d048635 Putative disease | plant-type hypersensitive response |
| | resistance RPP13-like protein 3-like | |
| | protein | |
| SEQ ID NO: 274 | Zm00001d006795 Omega- | transferase activity, transferring |
| | hydroxypalmitate O-feruloyl transferase | acyl groups other than amino-acyl |
| | | groups |
| SEQ ID NO: 275 | Zm00001d041972 Cellulose synthase-like | ellulose biosynthetic process |
| | protein G2-like protein | |
| SEQ ID NO: 276 | Zm00001d000183 Hexose carrier protein | carbohydrate transport |
| | HEX6-like sugar transport protein | |
| SEQ ID NO: 277 | Zm00001d039575 1-aminocyclopropane- | oxidoreductase activity, acting on |
| | 1-carboxylate oxidase | paired donors, with incorporation |
| | | or reduction of molecular oxygen, |
| | | 2-oxoglutarate as one donor, and |
| | | incorporation of one atom each of |
| | | oxygen into both donors |
| SEQ ID NO: 278 | Zm00001d046202 Transposase, | sequence-specific DNA binding |
| | Ptta/En/Spm, plant | |
| SEQ ID NO: 279 | Zm00001d013571 Ubiquitin-protein | protein binding |
| | ligase/zinc ion binding protein | |
| SEQ ID NO: 280 | Zm00001d015470 GDU1 | multicellular organism |
| | | development |
| SEQ ID NO: 281 | Zm00001d053965 16.9 kDa class I heat | response to stress |
| | shock protein 1 | |
| SEQ ID NO: 282 | Zm00001d029164 Inactive beta-amylase | ellular polysaccharide catabolic |
| | 9-like protein | process |
| SEQ ID NO: 283 | Zm00001d031717 Transcription factor | transcription, DNA-templated |
| | MYC4-like protein | |
| SEQ ID NO: 284 | Zm00001d035767 Jasmonate O- | jasmonic acid metabolic process |
| | methyltransferase | |
| SEQ ID NO: 285 | Zm00001d004279 Myrcene synthase, | monoterpenoid biosynthetic |
| | chloroplastic-like protein | process |
| SEQ ID NO: 286 | Zm00001d003462 Lipoyl synthase 2, | lipoate synthase activity |
| | mitochondrial-like protein | |
| SEQ ID NO: 287 | Zm00001d026360 Cyclin PHO80-like | protein kinase binding |
| | protein | |
| SEQ ID NO: 288 | Zm00001d013984 Protein EXECUTER 1 | 3′-5′-exoribonuclease activity |
| | (EXI) | |
| SEQ ID NO: 289 | Zm00001d047207 Copper-transporting | calcium-transporting ATPase |
| | ATPase (CTATP) | activity |
| SEQ ID NO: 290 | Zm00001d030381 Deoxyribodipyrimidine | protein-chromophore linkage |
| | photo-lyase; Photoreactivating enzyme | |
| | CPD photolyase | |
| SEQ ID NO: 291 | Zm00001d046755 Two-component | phosphorelay response regulator |
| | response regulator ARR12-like protein | activity |
| SEQ ID NO: 292 | Zm00001d007167 CBL-interacting | peptidyl-serine phosphorylation |
| | protein kinase 07 | |
| SEQ ID NO: 293 | Zm00001d039392 High affinity cationic | L-alpha-amino acid |
| | amino acid transporter | transmembrane transport |
| SEQ ID NO: 294 | Zm00001d013627 Retinoblastoma- | chromatin silencing by small RNA |
| | binding protein | |
| SEQ ID NO: 295 | Zm00001d044717 Cyclic nucleotide | ion gated channel activity |
| | binding/inward rectifier potassium | |
| | channel | |
| SEQ ID NO: 296 | Zm00001d032933 KIP1-like protein | N-acetyltransferase activity |
| SEQ ID NO: 297 | Zm00001d034467 Transcription regulator | cell differentiation |
| | HTH, Myb-type, DNA-binding protein | |
| SEQ ID NO: 298 | Zm00001d018797 Photosystem I reaction | photosynthesis |
| | center subunit psaK, chloroplast precursor | |
| SEQ ID NO: 299 | Zm00001d002934 Secondary wall NAC | sequence-specific DNA binding |
| | transcription factor 4 | |
| SEQ ID NO: 300 | Zm00001d022088 cDNA Agamous-like | DNA-binding transcription factor |
| | MADS-box protein AGL8 | activity |
| SEQ ID NO: 301 | Zm00001d023455 cDNA Transcription | DNA-binding transcription factor |
| | repressor OFP13 | activity |
| SEQ ID NO: 302 | Zm00001d023456 cDNA Transcription | DNA-binding transcription factor |
| | repressor OFP6 | activity |
| SEQ ID NO: 303 | Zm00001d038273 cDNA Coronatine- | shade avoidance |
| | insensitive protein 1 | |
| SEQ ID NO: 304 | Zm00001d004053 cDNA hypothetical | none |
| | protein | |
| SEQ ID NO: 305 | Zm00001d029183 cDNA cytochrome | isoflavone 2′-hydroxylase activity |
| | P450 family 81 subfamily D | |
| | polypeptide 8 | |
| SEQ ID NO: 306 | Zm00001d051194 cDNA Arginine | arginine decarboxylase activity |
| | decarboxylase | |
| SEQ ID NO: 307 | Zm00001d033132 cDNA photosystem II | photosynthesis |
| | light harvesting complex gene 2.1 | |
| SEQ ID NO: 308 | Zm00001d029215 cDNA Villin-2 | actin filament bundle assembly |
| SEQ ID NO: 309 | Zm00001d033543 cDNA Integral | cellular carbohydrate metabolic |
| | membrane HPP family protein | process |
| SEQ ID NO: 310 | Zm00001d053925 cDNA hypothetical | chlorophyll biosynthetic process |
| | protein | |
| SEQ ID NO: 311 | Zm00001d028269 cDNA hypothetical | DNA-binding transcription factor |
| | protein | activity |
| SEQ ID NO: 312 | Zm00001d033544 cDNA | transferase activity, transferring |
| | Glycosyltransferase family protein 64 | hexosyl groups |
| | protein C5 | |
| SEQ ID NO: 313 | Zm00001d034015 cDNA exoglucanase1 | glucan exo-1,3-beta-glucosidase |
| | | activity |
| SEQ ID NO: 314 | Zm00001d027743 cDNA | sugar mediated signaling pathway |
| | Serine/threonine-protein kinase EDR1 | |
| SEQ ID NO: 315 | Zm00001d048311 cDNA Sucrose | sucrose transmembrane transporter |
| | transport protein SUC3 | activity |
| SEQ ID NO: 316 | Zm00001d047256 cDNA Protein kinase | signal transduction |
| | domain superfamily protein | |
| SEQ ID NO: 317 | Zm00001d048720 cDNA Glutaredoxin- | electron transport chain |
| | C13 | |
| SEQ ID NO: 318 | Zm00001d023426 cDNA hypothetical | chlorophyll biosynthetic process |
| | protein | |
| SEQ ID NO: 319 | Zm00001d004331 cDNA hypothetical | regulation of transcription, DNA- |
| | protein | templated |
| SEQ ID NO: 320 | Zm00001d050748 cDNA proline-rich | ATPase activity, coupled to |
| | family protein | transmembrane movement of |
| | | substances |
| SEQ ID NO: 321 | Zm00001d010321 cDNA pyruvate | pyruvate, phosphate dikinase |
| | orthophosphate dikinase2 | activity |
| SEQ ID NO: 322 | Zm00001d004894 cDNA ribulose | ribulose-bisphosphate carboxylase |
| | bisphosphate carboxylase small subunit2 | activity |
| SEQ ID NO: 323 | Zm00001d042346 cDNA alpha/beta- | haloalkane dehalogenase activity |
| | Hydrolases superfamily protein | |
| SEQ ID NO: 324 | Zm00001d031657 cDNA putative | acid phosphatase activity |
| | inactive purple acid phosphatase 27 | |
| SEQ ID NO: 325 | Zm00001d008178 cDNA ABC | basipetal auxin transport |
| | transporter B family member 21 | |
| SEQ ID NO: 326 | Zm00001d043044 cDNA Secl/munc18- | kinase activity |
| | like (SM) proteins superfamily | |
| SEQ ID NO: 327 | Zm00001d018623 cDNA Photosynthetic | electron transporter, transferring |
| | NDH subunit of lumenal location 3 | electrons within the cyclic electron |
| | chloroplastic | transport pathway of |
| | | photosynthesis activity |
| SEQ ID NO: 328 | Zm00001d043095 cDNA Zinc finger | chloroplast organization |
| | protein VAR3 chloroplastic | |
| SEQ ID NO: 329 | Zm00001d029062 cDNA Vicilin-like | nutrient reservoir activity |
| | seed storage protein | |
| SEQ ID NO: 330 | Zm00001d011900 cDNA RNA-binding | RNA binding |
| | protein BRN1 | |
| SEQ ID NO: 331 | Zm00001d010672 cDNA Metacaspase | phosphoglycerate kinase activity |
| | type II | |
| SEQ ID NO: 332 | Zm00001d011183 cDNA thiamine | thiazole biosynthetic process |
| | biosynthesis1 | |
| SEQ ID NO: 333 | Zm00001d037103 cDNA Peroxiredoxin | peroxiredoxin activity |
| | Q chloroplastic | |
| SEQ ID NO: 334 | Zm00001d009028 cDNA Triose | transporter activity |
| | phosphate/phosphate translocator, | |
| | chloroplastic | |
| SEQ ID NO: 335 | Zm00001d013937 cDNA | chlorophyll biosynthetic process |
| | Protochlorophyllide reductase C | |
| | chloroplastic | |
| SEQ ID NO: 336 | Zm00001d002873 cDNA UPF0426 | plastoglobule |
| | protein chloroplastic | |
| SEQ ID NO: 337 | Zm00001d037362 cDNA DNA | DNA topoisomerase activity |
| | topoisomerase type IA core | |
| SEQ ID NO: 338 | Zm00001d026404 cDNA hypothetical | arginine catabolic process to |
| | protein | glutamate |
| SEQ ID NO: 339 | Zm00001d047255 cDNA 3-oxoacyl- | 3-oxoacyl-[acyl-carrier-protein] |
| | [acyl-carrier-protein] synthase II | synthase activity |
| | chloroplastic | |
| SEQ ID NO: 340 | Zm00001d031253 cDNA dicarboxylic | transporter activity |
| | acid transported | |
| SEQ ID NO: 341 | Zm00001d027576 cDNA hypothetical | voltage-gated anion channel |
| | protein | activity |
| SEQ ID NO: 342 | Zm00001d052595 cDNA Ribulose | ribulose-bisphosphate carboxylase |
| | bisphosphate carboxylase small chain, | activity |
| | chloroplastic | |
| SEQ ID NO: 343 | Zm00001d013367 cDNA Tubulin alpha-4 | structural molecule activity |
| | chain | |
| SEQ ID NO: 344 | Zm00001d046001 cDNA Triose | transmembrane transporter activity |
| | phosphate/phosphate translocator TPT | |
| | chloroplastic | |
| SEQ ID NO: 345 | Zm00001d008963 cDNA GDT1-like | chloroplast membrane |
| | protein 1 chloroplastic | |
| SEQ ID NO: 346 | Zm00001d048515 cDNA Stress | gene silencing |
| | responsive alpha-beta barrel domain | |
| | protein | |
| SEQ ID NO: 347 | Zm00001d018779 cDNA hypothetical | none |
| | protein | |
| SEQ ID NO: 348 | Zm00001d052595 cDNA Ribulose | ribulose-bisphosphate carboxylase |
| | bisphosphate carboxylase small chain, | activity |
| | chloroplastic | |
| SEQ ID NO: 349 | Zm00001d042049 cDNA Ferredoxin | photosynthetic electron transport |
| | | in photosystem I |
| SEQ ID NO: 350 | Zm00001d040242 cDNA Nuclear | cellular macromolecule metabolic |
| | transport factor 2 (NTF2) family protein | process |
| SEQ ID NO: 351 | Zm00001d042697 cDNA photosystem II | PSII associated light-harvesting |
| | subunit PsbS1 | complex II |
| SEQ ID NO: 352 | Zm00001d019518 cDNA Photosystem I | photosystem I reaction center |
| | reaction center subunit IV A | |
| SEQ ID NO: 353 | Zm00001d048313 cDNA NAD(P)-linked | photosystem II assembly |
| | oxidoreductase superfamily protein | |
| SEQ ID NO: 354 | Zm00001d033150 cDNA hypothetical | transcription regulatory region |
| | protein | sequence-specific DNA binding |
| SEQ ID NO: 355 | Zm00001d007858 cDNA Pyridoxal | oxidoreductase activity |
| | reductase chloroplastic | |
| SEQ ID NO: 356 | Zm00001d003588 cDNA Ras-related | protein binding |
| | protein RABD1 | |
| SEQ ID NO: 357 | Zm00001d036903 cDNA Plant Tudor-like | RNA binding |
| | RNA-binding protein | |
| SEQ ID NO: 358 | Zm00001d031484 cDNA PsbP domain- | photosystem II oxygen evolving |
| | containing protein 3 chloroplastic | complex |
| SEQ ID NO: 359 | Zm00001d018157 cDNA light harvesting | photosynthesis, light harvesting in |
| | complex a/b protein4 | photosystem I |
| SEQ ID NO: 360 | Zm00001d005814 cDNA Photosystem I | photosynthesis, light harvesting in |
| | chlorophyll a/b-binding protein 6 | photosystem I |
| | chloroplastic | |
| SEQ ID NO: 361 | Zm00001d033338 cDNA NAD(P)-linked | oxidoreductase activity |
| | oxidoreductase superfamily protein | |
| SEQ ID NO: 362 | Zm00001d053446 cDNA potassium | voltage-gated potassium channel |
| | channel3 | activity |
| SEQ ID NO: 363 | Zm00001d026603 cDNA Magnesium- | photosynthesis, light reaction |
| | chelatase subunit ChlH chloroplastic | |
| SEQ ID NO: 364 | Zm00001d027841 cDNA Ribulose- | pentose-phosphate shunt, non- |
| | phosphate 3-epimerase | oxidative branch |
| SEQ ID NO: 365 | Zm00001d030048 cDNA pfkB-like | isopentenyl diphosphate |
| | carbohydrate kinase family protein | biosynthetic process, |
| | | methylerythritol 4-phosphate |
| | | pathway |
| SEQ ID NO: 366 | Zm00001d005346 cDNA Aldo-keto | oxidoreductase activity |
| | reductase/oxidoreductase | |
| SEQ ID NO: 367 | Zm00001d023706 cDNA thioredoxin M1 | transcription coregulator activity |
| SEQ ID NO: 368 | Zm00001d042050 cDNA Protein | chlorophyll biosynthetic process |
| | RETICULATA-RELATED 4 | |
| | chloroplastic | |
| SEQ ID NO: 369 | Zm00001d042533 cDNA Trigger factor | protein transport |
| SEQ ID NO: 370 | Zm00001d022590 cDNA hypothetical | transcription, DNA-templated |
| | protein | |
| SEQ ID NO: 371 | Zm00001d045431 cDNA Enolase 1 | phosphopyruvate hydratase |
| | | activity |
| SEQ ID NO: 372 | Zm00001d047743 cDNA fatty acid | oxidoreductase activity, acting on |
| | desaturase7 | paired donors, with oxidation of a |
| | | pair of donors resulting in the |
| | | reduction of molecular oxygen to |
| | | two molecules of water |
| SEQ ID NO: 373 | Zm00001d036738 cDNA S-adenosyl-L- | methyltransferase activity |
| | methionine-dependent methyltransferase | |
| | superfamily protein | |
| SEQ ID NO: 374 | Zm00001d018274 cDNA Isoleucine- | valyl-tRNA aminoacylation |
| | tRNA ligase chloroplastic/mitochondrial | |
| SEQ ID NO: 375 | Zm00001d035003 cDNA ferredoxin2 | electron transfer activity |
| SEQ ID NO: 376 | Zm00001d027321 cDNA hypothetical | peptidyl-prolyl cis-trans isomerase |
| | protein | activity |
| SEQ ID NO: 377 | Zm00001d014284 cDNA CMV 1a | methyltransferase activity |
| | interacting protein 1 | |
| SEQ ID NO: 378 | Zm00001d036340 cDNA photosystem III | photosystem II |
| SEQ ID NO: 379 | Zm00001d003767 cDNA Photosystem I | photosynthesis, light harvesting in |
| | subunit O | photosystem I |
| SEQ ID NO: 380 | Zm00001d031997 cDNA NAD(P)- | nucleotide binding |
| | binding Rossmann-fold superfamily | |
| | protein | |
| SEQ ID NO: 381 | Zm00001d019479 cDNA granule-bound | NDP-glucose-starch |
| | starch synthase1b | glucosyltransferase activity |
| SEQ ID NO: 382 | Zm00001d024148 cDNA Photosynthetic | photosynthetic electron transport |
| | NDH subunit of subcomplex B 1 | in photosystem I |
| | chloroplastic | |
| SEQ ID NO: 383 | Zm00001d038579 cDNA | photosynthesis, light reaction |
| | Phosphoglycerate kinase | |
| SEQ ID NO: 384 | Zm00001d037273 cDNA Peptide | electron transport chain |
| | methionine sulfoxide reductase msrB | |
| SEQ ID NO: 385 | Zm00001d025845 cDNA hypothetical | chloroplast thylakoid membrane |
| | protein | |
| SEQ ID NO: 386 | Zm00001d027422 cDNA PsbP-like | chloroplast photosystem II |
| | protein 1 chloroplastic | |
| SEQ ID NO: 387 | Zm00001d024519 cDNA rubredoxin | iron ion binding |
| | family protein | |
| SEQ ID NO: 388 | Zm00001d040163 cDNA deoxy xylulose | 1-deoxy-D-xylulose-5-phosphate |
| | reductoisomerase1 | reductoisomerase activity |
| SEQ ID NO: 389 | Zm00001d012868 cDNA carotene | oxidoreductase activity |
| | isomerase3 | |
| SEQ ID NO: 390 | Zm00001d021763 cDNA photosystem II | photosynthesis, light harvesting in |
| | subunit29 | photosystem I |
| SEQ ID NO: 391 | Zm00001d035761 cDNA Peptidyl-prolyl | peptidyl-prolyl cis-trans isomerase |
| | cis-trans isomerase | activity |
| SEQ ID NO: 392 | Zm00001d032301 cDNA S-adenosyl-L- | methyltransferase activity |
| | methionine-dependent methyltransferase | |
| | superfamily protein | |
| SEQ ID NO: 393 | Zm00001d028562 cDNA Fructose-1,6- | photosynthetic electron transport |
| | bisphosphatase | in photosystem I |
| SEQ ID NO: 394 | Zm00001d034538 cDNA Rubredoxin-like | iron ion binding |
| | superfamily protein | |
| SEQ ID NO: 395 | Zm00001d048116 cDNA hypothetical | isopentenyl diphosphate |
| | protein | biosynthetic process, |
| | | methylerythritol 4-phosphate |
| | | pathway |
| SEQ ID NO: 396 | Zm00001d048998 cDNA Chlorophyll a-b | photosynthesis |
| | binding protein CP26 chloroplastic | |
| SEQ ID NO: 397 | Zm00001d049490 cDNA Protein | translation initiation factor activity |
| | CHLORORESPIRATORY REDUCTION | |
| | 6 chloroplastic | |
| SEQ ID NO: 398 | Zm00001d015975 cDNA putative | lactoylglutathione lyase activity |
| | lactoylglutathione lyase chloroplastic | |
| SEQ ID NO: 399 | Zm00001d021435 cDNA hypothetical | none |
| | protein | |
| SEQ ID NO: 400 | Zm00001d015613 cDNA Protein TIC 21 | protein import into chloroplast |
| | chloroplastic | stroma |
| SEQ ID NO: 401 | Zm00001d039258 cDNA Triose | glucose-6-phosphate |
| | phosphate/phosphate translocator TPT | transmembrane transporter activity |
| | chloroplastic | |
| SEQ ID NO: 402 | Zm00001d007267 cDNA light harvesting | photosynthesis, light harvesting in |
| | chlorophyll a/b binding protein5 | photosystem I |
| SEQ ID NO: 403 | Zm00001d033383 cDNA | thiamine biosynthetic process |
| | hydroxymethylpyrimidine phosphate | |
| | synthase1 | |
| SEQ ID NO: 404 | Zm00001d026645 cDNA K(+) efflux | solute: proton antiporter activity |
| | antiporter 2 chloroplastic | |
| SEQ ID NO: 405 | Zm00001d023757 cDNA NAD(P)H- | heat shock protein binding |
| | quinone oxidoreductase subunit U | |
| | chloroplastic | |
| SEQ ID NO: 406 | Zm00001d034005 cDNA evolutionarily | oxidation-reduction process |
| | conserved C-terminal region 2 | |
| SEQ ID NO: 407 | Zm00001d022381 cDNA NADPH- | hydrogen peroxide catabolic |
| | dependent thioredoxin reductase 3 | process |
| SEQ ID NO: 408 | Zm00001d031962 cDNA hypothetical | protein dephosphorylation |
| | protein | |
| SEQ ID NO: 409 | Zm00001d005446 cDNA Photosystem I | photosystem I reaction center |
| | reaction center subunit IV A | |
| SEQ ID NO: 410 | Zm00001d027511 cDNA Catalase | hydrogen peroxide catabolic |
| | isozyme 2 | process |
| SEQ ID NO: 411 | Zm00001d017178 cDNA hypothetical | primary metabolic process |
| | protein | |
| SEQ ID NO: 412 | Zm00001d03 8947 cDNA putative | electron transfer activity |
| | galacturonosyltransferase-like 9 | |
| SEQ ID NO: 413 | Zm00001d034739 cDNA | acetylpyruvate hydrolase activity |
| | Fumarylacetoacetate (FAA) hydrolase | |
| | family | |
| SEQ ID NO: 414 | Zm00001d044745 cDNA Alanine--tRNA | alanyl-tRNA aminoacylation |
| | ligase chloroplastic/mitochondrial | |
| SEQ ID NO: 415 | Zm00001d038491 cDNA | N-acetyltransferase activity |
| | Acetyltransferase NSI | |
| SEQ ID NO: 416 | Zm00001d014445 cDNA Protein kinase | kinase activity |
| | superfamily protein | |
| SEQ ID NO: 417 | Zm00001d039745 cDNA Protein | phosphoprotein phosphatase |
| | phosphatase 2C | activity |
| SEQ ID NO: 418 | Zm00001d038485 cDNA | photosystem II stabilization |
| | Serine/threonine-protein kinase STN8 | |
| | chloroplastic | |
| SEQ ID NO: 419 | Zm00001d012287 cDNA hypothetical | starch biosynthetic process |
| | protein | |
| SEQ ID NO: 420 | Zm00001d027694 cDNA Solanesyl | plastoquinone biosynthetic process |
| | diphosphate synthase 2 chloroplastic | |
| SEQ ID NO: 421 | Zm00001d003470 cDNA putative plastid- | response to abscisic acid |
| | lipid-associated protein 2 chloroplastic | |
| SEQ ID NO: 422 | Zm00001d019180 cDNA Nudix | hydrolase activity |
| | hydrolase 16 mitochondrial | |
| SEQ ID NO: 423 | Zm00001d007394 cDNA Rubredoxin-like | iron ion binding |
| | superfamily protein | |
| SEQ ID NO: 424 | Zm00001d053432 cDNA iron-sulfur | electron transporter, transferring |
| | protein2 | electrons within cytochrome b6/f |
| | | complex of photosystem II activity |
| SEQ ID NO: 425 | Zm00001d039900 cDNA putative zinc | metalloendopeptidase activity |
| | metalloprotease EGY2 chloroplastic | |
| SEQ ID NO: 426 | Zm00001d050810 cDNA 2-hydroxy-3- | pentose-phosphate shunt |
| | oxopropionate reductase | |
| SEQ ID NO: 427 | Zm00001d016802 cDNA L-ascorbate | L-ascorbate peroxidase activity |
| | peroxidase S chloroplastic/mitochondrial | |
| SEQ ID NO: 428 | Zm00001d004978 cDNA Saccharopine | oxidoreductase activity |
| | dehydrogenase | |
| SEQ ID NO: 429 | Zm00001d028924 cDNA NifU-like | iron-sulfur cluster assembly |
| | protein 1 chloroplastic | |
| SEQ ID NO: 430 | Zm00001d002815 cDNA NAD(P)H- | oxidoreductase activity, acting on |
| | quinone oxidoreductase subunit M | NAD(P)H, quinone or similar |
| | chloroplastic | compound as acceptor |
| SEQ ID NO: 431 | Zm00001d029065 cDNA Protein LRP16 | regulation of transcription, DNA- |
| | | templated |
| SEQ ID NO: 432 | Zm00001d052184 cDNA Protein | chloroplast envelope |
| | CURVATURE THYLAKOID 1D | |
| | chloroplastic | |
| SEQ ID NO: 433 | Zm00001d038337 cDNA DAR GTPase 3 | GTP binding |
| | chloroplastic | |
| SEQ ID NO: 434 | Zm00001d003713 cDNA Exocyst | pollen tube growth |
| | complex component SEC5A | |
| SEQ ID NO: 435 | Zm00001d031953 cDNA Thioredoxin | protein disulfide oxidoreductase |
| | family protein | activity |
| SEQ ID NO: 436 | Zm00001d053576 cDNA putative | eukaryotic translation elongation |
| | elongation factor 1-gamma 2 | factor 1 complex |
| SEQ ID NO: 437 | Zm00001d000123 cDNA NAD(P)- | response to oxidative stress |
| | binding Rossmann-fold superfamily | |
| | protein | |
| SEQ ID NO: 438 | Zm00001d045431 cDNA Enolase 1 | phosphopyruvate hydratase |
| | | activity |
| SEQ ID NO: 439 | Zm00001d036630 cDNA Filamentation | metalloendopeptidase activity |
| | temperature-sensitive H 2B | |
| SEQ ID NO: 440 | Zm00001d048515 cDNA Stress | gene silencing |
| | responsive alpha-beta barrel domain | |
| | protein | |
| SEQ ID NO: 441 | Zm00001d021310 cDNA | reductive pentose-phosphate cycle |
| | Triosephosphate isomerase | |
| SEQ ID NO: 442 | Zm00001d042353 cDNA sucrose | sucrose synthase activity |
| | phosphate synthase2 | |
| SEQ ID NO: 443 | Zm00001d050150 cDNA Adenylate | adenylate kinase activity |
| | kinase 5 chloroplastic | |
| SEQ ID NO: 444 | Zm00001d025545 cDNA zeaxanthin | abscisic acid biosynthetic process |
| | epoxidase2 | |
| SEQ ID NO: 445 | Zm00001d012168 cDNA Membrane- | chloroplast thylakoid membrane |
| | associated protein VIPP1 chloroplastic | |
| SEQ ID NO: 446 | Zm00001d011581 cDNA Peroxiredoxin- | peroxiredoxin activity |
| | 2B | |
| SEQ ID NO: 447 | Zm00001d018030 cDNA Photosynthetic | regulation of transcription by RNA |
| | NDH subunit of subcomplex B 4 | polymerase II |
| | chloroplastic | |
| SEQ ID NO: 448 | Zm00001d018145 cDNA Presequence | protein processing |
| | protease 2 chloroplastic/mitochondrial | |
| SEQ ID NO: 449 | Zm00001d040221 cDNA Peptidase | metalloendopeptidase activity |
| | family M48 family protein | |
| SEQ ID NO: 450 | Zm00001d033594 cDNA Myelin- | phosphorus metabolic process |
| | associated oligodendrocyte basic protein | |
| | isoform 1 | |
| SEQ ID NO: 451 | Zm00001d008625 cDNA Inner membrane | thylakoid membrane organization |
| | protein ALBINO3 chloroplastic | |
| SEQ ID NO: 452 | Zm00001d032380 cDNA acclimation of | photosystem II assembly |
| | photosynthesis to environment | |
| SEQ ID NO: 453 | Zm00001d045575 cDNA Ferredoxin- | photosynthetic electron transport |
| | NADP reductase leaf isozyme 1 | in photosystem I |
| | chloroplastic | |
| SEQ ID NO: 454 | Zm00001d042526 cDNA Rhodanese/Cell | regulation of stomatai closure |
| | cycle control phosphatase superfamily | |
| | protein | |
| SEQ ID NO: 455 | Zm00001d043168 cDNA MAR-binding | photosystem II assembly |
| | filament-like protein 1-1 isoform 2 | |
| SEQ ID NO: 456 | Zm00001d053545 cDNA NAD(P)- | nucleotide binding |
| | binding Rossmann-fold superfamily | |
| | protein | |
| SEQ ID NO: 457 | Zm00001d053981 cDNA putative | pyridoxal phosphate biosynthetic |
| | pyridoxal 5′-phosphate synthase subunit | process |
| | PDX2 | |
| SEQ ID NO: 458 | Zm00001d017746 cDNA vitamin E | tocopherol O-methyltransferase |
| | synthesis4 | activity |
| SEQ ID NO: 459 | Zm00001d012083 cDNA Thioredoxin F- | pentose-phosphate shunt |
| | type chloroplastic | |
| SEQ ID NO: 460 | Zm00001d024718 cDNA Beta-carotene | strigolactone biosynthetic process |
| | isomerase D27 chloroplastic | |
| SEQ ID NO: 461 | Zm00001d021621 cDNA Polynucleotidyl | 3′-5′ exonuclease activity |
| | transferase ribonuclease H fold protein | |
| | with HRDC domain | |
| SEQ ID NO: 462 | Zm00001d015004 cDNA Protein LOW | aromatic amino acid family |
| | PSII ACCUMULATION 3 chloroplastic | biosynthetic process |
| SEQ ID NO: 463 | Zm00001d039131 cDNA ADP glucose | glucose-1-phosphate |
| | pyrophosphorylase2 | adenylyltransferase activity |
| SEQ ID NO: 464 | Zm00001d044970 cDNA putative | protein tyrosine phosphatase |
| | tyrosine-protein phosphatase | activity |
| SEQ ID NO: 465 | Zm00001d018401 cDNA plastid | positive regulation of ubiquitin |
| | transcriptionally active 17 | protein ligase activity |
| SEQ ID NO: 466 | Zm00001d015975 cDNA putative | lactoylglutathione lyase activity |
| | lactoylglutathione lyase chloroplastic | |
| SEQ ID NO: 467 | Zm00001d013428 cDNA | phosphoglucomutase activity |
| | phosphoglucomutase2 | |
| SEQ ID NO: 468 | Zm00001d021246 cDNA Ras-related | chloroplast thylakoid membrane |
| | protein RABA3 | |
| SEQ ID NO: 469 | Zm00001d007921 cDNA Tic62 protein | oxidation-reduction process |
| SEQ ID NO: 470 | Zm00001d027511 cDNA Catalase | hydrogen peroxide catabolic |
| | isozyme 2 | process |
| SEQ ID NO: 471 | Zm00001d027309 cDNA Phosphoglucan | intracellular signal transduction |
| | phosphatase DSP4 chloroplastic | |
| SEQ ID NO: 472 | Zm00001d025544 cDNA Zeaxanthin | zeaxanthin epoxidase [overall] |
| | epoxidase chloroplastic | activity |
| SEQ ID NO: 473 | Zm00001d039276 cDNA Ubiquitin ligase | cellular metabolic process |
| | SINAT3 | |
| SEQ ID NO: 474 | Zm00001d003512 cDNA Zeaxanthin | zeaxanthin epoxidase [overall] |
| | epoxidase chloroplastic | activity |
| SEQ ID NO: 475 | Zm00001d053861 cDNA Putative | GTP binding |
| | translation elongation factor family | |
| | protein | |
| SEQ ID NO: 476 | Zm00001d038894 cDNA | circadian rhythm |
| | Serine/threonine-protein kinase STN7 | |
| | chloroplastic | |
| SEQ ID NO: 477 | Zm00001d051321 cDNA ATP-dependent | metalloendopeptidase activity |
| | zinc metalloprotease FTSH 7 | |
| | chloroplastic | |
| SEQ ID NO: 478 | Zm00001d016826 cDNA proline-rich | anatomical structure |
| | family protein | morphogenesis |
| SEQ ID NO: 479 | Zm00001d016854 cDNA Ferrochelatase | starch biosynthetic process |
| SEQ ID NO: 480 | Zm00001d017435 cDNA hypothetical | none |
| | protein | |
| SEQ ID NO: 481 | Zm00001d048373 cDNA Carotenoid | oxidoreductase activity, acting on |
| | 910(9′10′)-cleavage dioxygenase 1 | single donors with incorporation of |
| | | molecular oxygen, incorporation |
| | | of two atoms of oxygen |
| SEQ ID NO: 482 | Zm00001d018901 cDNA protein | protein binding |
| | containing PDZ domain a K-box domain | |
| | and a TPR region | |
| SEQ ID NO: 483 | Zm00001d019454 cDNA PGR5-like | photosynthetic electron transport |
| | protein 1B chloroplastic | in photosystem I |
| SEQ ID NO: 484 | Zm00001d031899 cDNA malate | malate dehydrogenase (NADP+) |
| | dehydrogenase6 | activity |
| SEQ ID NO: 485 | Zm00001d045706 cDNA Restorer of | aldehyde dehydrogenase (NAD) |
| | fertility2 | activity |
| SEQ ID NO: 486 | Zm00001d035869 cDNA Protein MEI2- | nucleotide binding |
| | like 5 | |
| SEQ ID NO: 487 | Zm00001d053262 cDNA calcium- | protein localization |
| | dependent lipid-binding family protein | |
| SEQ ID NO: 488 | Zm00001d017958 cDNA Glutamine | glutamine biosynthetic process |
| | synthetase root isozyme 3 | |
| SEQ ID NO: 489 | Zm00001d007113 cDNA Xylose | xylose isomerase activity |
| | isomerase | |
| SEQ ID NO: 490 | Zm00001d051650 cDNA Sucrose | transcription, DNA-templated |
| | cleavage protein-like protein | |
| SEQ ID NO: 491 | Zm00001d015138 cDNA hypothetical | nuclear pore |
| | protein | |
| SEQ ID NO: 492 | Zm00001d030103 cDNA putative | xyloglucamxyloglucosyl |
| | xyloglucan endotransglucosylase/ | transferase activity |
| | hydrolase protein 30 | |
| SEQ ID NO: 493 | Zm00001d021846 cDNA Insulin- | metalloendopeptidase activity |
| | degrading enzyme-like 1 peroxisomal | |
| SEQ ID NO: 494 | Zm00001d041576 cDNA Transcription | regulation of transcription, DNA- |
| | factor MYB48 | templated |
| SEQ ID NO: 495 | Zm00001d005391 cDNA Cysteine | cysteine-type endopeptidase |
| | proteinases superfamily protein | activity |
| SEQ ID NO: 496 | Zm00001d009022 cDNA NmrA-like | 166 nucleotide binding |
| | negative transcriptional regulator family | |
| | protein | |
| SEQ ID NO: 497 | Zm00001d041343 cDNA putative disease | defense response |
| | resistance protein | |
| SEQ ID NO: 498 | Zm00001d039965 cDNA Delta(7)-sterol- | fatty acid biosynthetic process |
| | C5(6)-desaturase 1 | |
| SEQ ID NO: 499 | Zm00001d029047 cDNA Receptor-like | signaling receptor activity |
| | protein kinase FERONIA | |
| SEQ ID NO: 500 | Zm00001d028230 cDNA Sugar transport | sucrose transport |
| | protein 13 | |
| SEQ ID NO: 501 | Zm00001d013243 cDNA Cyclic | plant-type hypersensitive response |
| | nucleotide-gated ion channel 2 | |
| SEQ ID NO: 502 | Zm00001d032926 cDNA chlorophyllase2 | chlorophyll catabolic process |
| SEQ ID NO: 503 | Zm00001d045667 cDNA Protein NRT1/ | nitrate assimilation |
| | PTR FAMILY 3.1 | |
| SEQ ID NO: 504 | Zm00001d021846 cDNA Insulin- | metalloendopeptidase activity |
| | degrading enzyme-like 1 peroxisomal | |
| SEQ ID NO: 505 | Zm00001d033469 cDNA Ferredoxin | electron transfer activity |
| SEQ ID NO: 506 | Zm00001d019563 cDNA Aquaporin | transporter activity |
| | PIP2-1 | |
| SEQ ID NO: 507 | Zm00001d003866 cDNA hypothetical | cell wall organization |
| | protein | |
| SEQ ID NO: 508 | Zm00001d039325 cDNA Putative | signaling receptor activity |
| | inactive receptor-like protein kinase | |
| SEQ ID NO: 509 | Zm00001d023516 cDNA Salt stress- | mannose binding |
| | induced protein | |
| SEQ ID NO: 510 | Zm00001d050918 cDNA hypothetical | aldehyde dehydrogenase (NAD) |
| | protein | activity |
| SEQ ID NO: 511 | Zm00001d025253 cDNA hypothetical | none |
| | protein | |
| SEQ ID NO: 512 | Zm00001d024499 cDNA Nuclear factor 1 | aminoacyl-tRNA hydrolase |
| | A-type isoform 2 | activity |
| SEQ ID NO: 513 | Zm00001d045948 cDNA Protein | drug transmembrane transporter |
| | DETOXIFICATION 16 | activity |
| SEQ ID NO: 514 | Zm00001d021569 cDNA Transparent | drug transmembrane transporter |
| | testa 12 protein | activity |
| SEQ ID NO: 515 | Zm00001d053327 cDNA Galactoside 2- | xyloglucan biosynthetic process |
| | alpha-L-fucosyltransferase | |
| SEQ ID NO: 516 | Zm00001d047208 cDNA WAT1-related | auxin-activated signaling pathway |
| | protein | |
| SEQ ID NO: 517 | Zm00001d007687 cDNA Tropinone | response to karrikin |
| | reductase-like protein | |
| SEQ ID NO: 518 | Zm00001d015126 cDNA response to low | pyridoxamine-phosphate oxidase |
| | sulfur 3 | activity |
| SEQ ID NO: 519 | Zm00001d034781 cDNA G-type lectin S- | multicellular organism |
| | receptor-like serine/threonine-protein | development |
| | kinase | |
| SEQ ID NO: 520 | Zm00001d016655 cDNA hypothetical | voltage-gated potassium channel |
| | protein | activity |
| SEQ ID NO: 521 | Zm00001d043517 cDNA Peptidase M28 | regulation of inflorescence |
| | family protein | meristem growth |
| SEQ ID NO: 522 | Zm00001d045667 cDNA Protein NRT1/ | nitrate assimilation |
| | PTR FAMILY 3.1 | |
| SEQ ID NO: 523 | Zm00001d000126 cDNA Function | voltage-gated potassium channel |
| | unknown | activity |
| SEQ ID NO: 524 | Zm00001d029706 cDNA Glutathione S- | glutathione transferase activity |
| | transferase GSTU6-like protein | |
| SEQ ID NO: 525 | Zm00001d029321 cDNA Cationic amino | amino acid transmembrane |
| | acid transporter | transporter activity |
| SEQ ID NO: 526 | Zm00001d014701 cDNA Transcription | regulation of transcription, DNA- |
| | regulator HTH, Myb-type, DNA-binding | templated |
| | protein | |
| SEQ ID NO: 527 | Zm00001d049624 cDNA Glutamate-rich | zinc ion transmembrane transport |
| | WD repeat-containing protein 1-like | |
| | protein | |
| SEQ ID NO: 528 | Zm00001d034359 cDNA Zinc finger, | nucleic acid binding |
| | C2H2-type/integrase, DNA-binding | |
| | protein | |
| SEQ ID NO: 529 | Zm00001d033924 cDNA Cell wall | proton-transporting ATP synthase |
| | protein pherophorin-C10 (PHC10) | activity, rotational mechanism |
| SEQ ID NO: 530 | Zm00001d032222 cDNA UDP- | glucuronosyltransferase activity |
| | glycosyltransferase 85A2-like protein | |
| SEQ ID NO: 531 | Zm00001d018155 cDNA Galactoside 2- | cell wall biogenesis |
| | alpha-L-fucosyltransferase-like protein | |
| SEQ ID NO: 532 | Zm00001d036263 cDNA Receptor | protein serine/threonine kinase |
| | protein serine/threonine kinase | activity |
| SEQ ID NO: 533 | Zm00001d044043 cDNA Acetylajmaline | lipid catabolic process |
| | esterase | |
| SEQ ID NO: 534 | Zm00001d027425 cDNA MADS-box | transcription, DNA-templated |
| | transcription factor 56-like protein | |
| SEQ ID NO: 535 | Zm00001d048635 cDNA Putative disease | plant-type hypersensitive response |
| | resistance RPP13-like protein 3-like | |
| | protein | |
| SEQ ID NO: 536 | Zm00001d006795 cDNA Omega- | transferase activity, transferring |
| | hydroxypalmitate O-feruloyl transferase | acyl groups other than amino-acyl |
| | | groups |
| SEQ ID NO: 537 | Zm00001d041972 cDNA Cellulose | ellulose biosynthetic process |
| | synthase-like protein G2-like protein | |
| SEQ ID NO: 538 | Zm00001d000183 cDNA Hexose carrier | carbohydrate transport |
| | protein HEX6-like sugar transport protein | |
| SEQ ID NO: 539 | Zm00001d039575 cDNA 1- | oxidoreductase activity, acting on |
| | aminocyclopropane-1-carboxylate oxidase | paired donors, with incorporation |
| | | or reduction of molecular oxygen, |
| | | 2-oxoglutarate as one donor, and |
| | | incorporation of one atom each of |
| | | oxygen into both donors |
| SEQ ID NO: 540 | Zm00001d046202 cDNA Transposase, | sequence-specific DNA binding |
| | Ptta/En/Spm, plant | |
| SEQ ID NO: 541 | Zm00001d013571 cDNA Ubiquitin- | protein binding |
| | protein ligase/zinc ion binding protein | |
| SEQ ID NO: 542 | Zm00001d015470 cDNA GDU1 | multicellular organism |
| | | development |
| SEQ ID NO: 543 | Zm00001d053965 cDNA 16.9 kDa class I | response to stress |
| | heat shock protein 1 | |
| SEQ ID NO: 544 | Zm00001d029164 cDNA Inactive beta- | ellular polysaccharide catabolic |
| | amylase 9-like protein | process |
| SEQ ID NO: 545 | Zm00001d031717 cDNA Transcription | transcription, DNA-templated |
| | factor MYC4-like protein | |
| SEQ ID NO: 546 | Zm00001d035767 cDNA Jasmonate O- | jasmonic acid metabolic process |
| | methyltransferase | |
| SEQ ID NO: 547 | Zm00001d004279 cDNA Myrcene | monoterpenoid biosynthetic |
| | synthase, chloroplastic-like protein | process |
| SEQ ID NO: 548 | Zm00001d003462 cDNA Lipoyl synthase | lipoate synthase activity |
| | 2, mitochondrial-like protein | |
| SEQ ID NO: 549 | Zm00001d026360 cDNA Cyclin PHO80- | protein kinase binding |
| | like protein | |
| SEQ ID NO: 550 | Zm00001d013984 cDNA Protein | 3′-5′-exoribonuclease activity |
| | EXECUTER 1 (EX1) | |
| SEQ ID NO: 551 | Zm00001d047207 cDNA Copper- | calcium-transporting ATPase |
| | transporting ATPase (CTATP) | activity |
| SEQ ID NO: 552 | Zm00001d030381 | protein-chromophore linkage |
| | cDNA Deoxyribodipyrimidine photo- | |
| | lyase; Photoreactivating enzyme CPD | |
| | photolyase | |
| SEQ ID NO: 553 | Zm00001d046755 cDNA Two- | phosphorelay response regulator |
| | component response regulator ARR12- | activity |
| | like protein | |
| SEQ ID NO: 554 | Zm00001d007167 cDNA CBL-interacting | peptidyl-serine phosphorylation |
| | protein kinase 07 | |
| SEQ ID NO: 555 | Zm00001d039392 cDNA High affinity | L-alpha-amino acid |
| | cationic amino acid transporter | transmembrane transport |
| SEQ ID NO: 556 | Zm00001d013627 | chromatin silencing by small RNA |
| | cDNA Retinoblastoma-binding protein | |
| SEQ ID NO: 557 | Zm00001d044717 cDNA Cyclic | ion gated channel activity |
| | nucleotide binding/inward rectifier | |
| | potassium channel | |
| SEQ ID NO: 558 | Zm00001d032933 cDNA KIP1-like | N-acetyltransferase activity |
| | protein | |
| SEQ ID NO: 559 | Zm00001d034467 cDNA Transcription | cell differentiation |
| | regulator HTH, Myb-type, DNA-binding | |
| | protein | |
| SEQ ID NO: 560 | Zm00001d018797 cDNA Photosystem I | photosynthesis |
| | reaction center subunit psaK, chloroplast | |
| | precursor | |
| SEQ ID NO: 561 | Zm00001d002934 cDNA Secondary wall | sequence-specific DNA binding |
| | NAC transcription factor 4 | |
| SEQ ID NO: 562 | Zmm28 | Transcription factor |
| SEQ ID NO: 563 | Zm00001d003911 (AFB2-like/TIR1-like | Auxin receptor |
| | (afb2)) | |
| SEQ ID NO: 564 | Zm00001d003911 genomic (AFB2- | Auxin receptor |
| | like/TIR1-like (afb2)) | |
| SEQ ID NO: 565 | AT5G24800.1, BASIC LEUCINE | Transcription factor |
| | ZIPPER 9 | |
| SEQ ID NO: 566 | AT5G24800.1, BASIC LEUCINE | Transcription factor |
| | ZIPPER 9 | |
| SEQ ID NO: 567 | Zm00001d051684, CONSTANS gene | Transcription factor |
| | family like 14 | |
| SEQ ID NO: 568 | Zm00001d044194, MYB-LIKE DNA- | Transcription factor |
| | BINDING PROTEIN, mybr97 - MYB- | |
| | related-transcription factor 97 | |
| SEQ ID NO: 569 | Zm00001d028974, ETHYLENE | Transcription factor |
| | INSENSITIVE 3-LIKE 1 | |
| SEQ ID NO: 570 | Zm00001d044301, PROTEIN | Signal transduction |
| | PHOSPHATASE 2C | |
| SEQ ID NO: 571 | Zm00001d015239, LANOSTEROL | Steroid biosynthetic process |
| | SYNTHASE | |
| SEQ ID NO: 573 | Zm00001d023933, 2,4-dihydroxy-1,4- | Response to wounding |
| | benzoxazin-3-one-glucoside dioxygenase/ | |
| | DIBOA-Glc dioxygenase | |
| SEQ ID NO: 573 | Zm00001d029875, ZmMYBR43, Myb- | Transcription factor |
| | like DNA-binding domain (Myb_DNA- | |
| | binding) | |
| SEQ ID NO: 574 | Zm00001d051684, CONSTANS gene | Transcription factor |
| | family like 14 | |
| SEQ ID NO: 575 | Zm00001d044194, MYB-LIKE DNA- | Transcription factor |
| | BINDING PROTEIN, mybr97 - MYB- | |
| | related-transcription factor 97 | |
| SEQ ID NO: 576 | Zm00001d028974, ETHYLENE | Transcription factor |
| | INSENSITIVE 3-LIKE 1 | |
| SEQ ID NO: 577 | Zm00001d044301, PROTEIN | Signal transduction |
| | PHOSPHATASE 2C | |
| SEQ ID NO: 578 | Zm00001d015239, LANOSTEROL | Steroid biosynthetic process |
| | SYNTHASE | |
| SEQ ID NO: 579 | Zm00001d023933, 2,4-dihydroxy-1,4- | Response to wounding |
| | benzoxazin-3-one-glucoside dioxygenase/ | |
| | DIBOA-Glc dioxygenase | |

### DETAILED DESCRIPTION

### I. Compositions

### A. Polynucleotides and Polypeptides

The present disclosure provides polynucleotides encoding polypeptides. Accordingly, as used herein “polypeptide,” “protein,” or the like, refers to a protein represented by a SEQ ID NO.

One aspect of the disclosure provides a polynucleotide encoding a polypeptide comprising an amino acid sequence that is at least 80-99% identical to the amino acid sequence of any one of SEQ ID NOS: 1-11, 23-31, 40-299, 563, 565, and 567-573).

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98).

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, California, GAP, BESTFIT, BLAST, FASTA and TFASTA in the Wisconsin Genetics Software Package®, Version 8 (available from Genetics Computer Group (GCG® programs (Accelrys, Inc., San Diego, Calif.)). The CLUSTAL program is well described by Higgins and Sharp, (1988) Gene 73:237 44; Higgins and Sharp, (1989) CABIOS 5:151 3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

Accordingly, in any of the embodiments described herein, the polynucleotide may encode a polypeptide that is at least 80% identical to any one of SEQ ID NOS: 1-11, 23-31, 40-299, 563, 565, and 567-573. For example, the polynucleotide may encode a polypeptide that is at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, or at least 99% identical to the amino acid sequence of any one of SEQ ID NOS: 1-11, 23-31, 40-299, 563, 565, and 567-573.

### B. Recombinant DNA Construct

Also provided is a recombinant DNA construct comprising any of the polynucleotides described herein. In certain embodiments, the recombinant DNA construct further comprises at least one regulatory element. In certain embodiments, the at least one regulatory element of the recombinant DNA construct comprises a promoter. In certain embodiments, the promoter is a heterologous promoter.

As used herein, a “recombinant DNA construct” comprises two or more operably linked DNA segments, preferably DNA segments that are not operably linked in nature (i.e., heterologous). Non-limiting examples of recombinant DNA constructs include a polynucleotide of interest operably linked to heterologous sequences, also referred to as “regulatory elements,” which aid in the expression, autologous replication, and/or genomic insertion of the sequence of interest. Such regulatory elements include, for example, promoters, termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The polynucleotides described herein can be provided in expression cassettes for expression in a plant of interest or any organism of interest. The cassette can include 5′ and 3′ regulatory sequences operably linked to a polynucleotide. “Operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (e.g., a promoter), a polynucleotide, and a transcriptional and translational termination region (e.g., termination region) functional in plants. The regulatory regions (e.g., promoters, transcriptional regulatory regions, and translational termination regions) and/or the polynucleotide may be native/analogous to the host cell or to each other. Alternatively, the regulatory regions and/or the polynucleotide may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, with the plant host, or may be derived from another source (i.e., foreign or heterologous) than the promoter, the polynucleotide, the plant host, or any combination thereof.

The expression cassette may additionally contain a 5′ leader sequences. Such leader sequences can act to enhance translation. Translation leaders are known in the art and include viral translational leader sequences.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein “promoter” refers to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Certain types of promoters preferentially initiate transcription in certain tissues, such as leaves, roots, seeds, fibers, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026); GOS2 (U.S. Pat. No. 6,504,083), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Also contemplated are synthetic promoters which include a combination of one or more heterologous regulatory elements.

### C. Plants and Plant Cells

Provided are plants, plant cells, plant parts, seed, and grain comprising a polynucleotide sequence described herein or a recombinant DNA construct described herein, so that the plants, plant cells, plant parts, seed, and/or grain have increased expression of a polypeptide. In certain embodiments, the plants, plant cells, plant parts, seeds, and/or grain have stably incorporated an exogenous polynucleotide described herein into its genome. In certain embodiments, the plants, plant cells, plant parts, seeds, and/or grain can comprise multiple polynucleotides (i.e., at least 1, 2, 3, 4, 5, 6 or more).

In specific embodiments, the polynucleotide(s) in the plants, plant cells, plant parts, seeds, and/or grain are operably linked to a heterologous regulatory element, such as, but not limited to, a constitutive promoter, a tissue-preferred promoter, or a synthetic promoter for expression in plants or a constitutive enhancer. For example, in certain embodiments the heterologous regulatory element is the maize GOS2 promoter.

Also provided herein are plants, plant cells, plant parts, seeds, and grain comprising an introduced genetic modification at a genomic locus that encodes a polypeptide comprising an amino acid sequence that is at least 80% identical to an amino acid sequence selected from the group consisting of SEQ ID NOS: 1-11, 23-31, 40-299, 563, 565, and 567-573.

In certain embodiments, the genetic modification increases the activity of the protein. In certain embodiments, the genetic modification increases the level of the protein. In certain embodiments, the genetic modification increases both the level and activity of the protein.

A “genomic locus” as used herein, generally refers to the location on a chromosome of the plant where a gene, such as a polynucleotide encoding a polypeptide, is found. As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

A “regulatory element” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located.

An “enhancer” element is any nucleic acid molecule that increases transcription of a nucleic acid molecule when functionally linked to a promoter regardless of its relative position.

A “repressor” (also sometimes called herein silencer) is defined as any nucleic acid molecule which inhibits the transcription when functionally linked to a promoter regardless of relative position.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Genetic modification,” “DNA modification,” and the like refers to a site-specific modification that alters or changes the nucleotide sequence at a specific genomic locus of the plant. The genetic modification of the compositions and methods described herein may be any modification known in the art such as, for example, insertion, deletion, single nucleotide polymorphism (SNP), and or a polynucleotide modification. Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region.

As used herein, a “targeted” genetic modification or “targeted” DNA modification, refers to the direct manipulation of an organism's genes. The targeted modification may be introduced using any technique known in the art, such as, for example, plant breeding, genome editing, or single locus conversion.

The type and location of the DNA modification of the polynucleotide is not particularly limited so long as the DNA modification results in increased expression and/or activity of the protein encoded by the corresponding polynucleotide.

In certain embodiments, the plant, plant cells, plant parts, seeds, and/or grain comprise one or more nucleotide modifications present within (a) the coding region; (b) non-coding region; (c) regulatory sequence; (d) untranslated region, or (e) any combination of (a)-(d) of an endogenous polynucleotide encoding a polypeptide.

In certain embodiments the DNA modification is an insertion of one or more nucleotides, preferably contiguous, in the genomic locus. For example, the insertion of an expression modulating element (EME), such as an EME described in PCT/US2018/025446, in operable linkage with the gene of interest described herein. In certain embodiments, the targeted DNA modification may be the replacement of an endogenous promoter with another promoter known in the art to have higher expression, such as, for example, the maize GOS2 promoter. In certain embodiments, the targeted DNA modification may be the insertion of a promoter known in the art to have higher expression, such as, for example, the maize GOS2 promoter, into the 5′UTR so that expression of the endogenous polypeptide is controlled by the inserted promoter. In certain embodiments, the DNA modification is a modification to optimize Kozak context to increase expression. In certain embodiments, the DNA modification is a polynucleotide modification or SNP at a site that regulates the stability of the expressed protein.

As used herein “increased,” “increase,” or the like refers to any detectable increase in an experimental group (e.g., plant with a DNA modification described herein) as compared to a control group (e.g., wild-type plant that does not comprise the DNA modification). Accordingly, increased expression of a protein comprises any detectable increase in the total level of the protein in a sample and can be determined using routine methods in the art such as, for example, Western blotting and ELISA.

In certain embodiments, the genomic locus has more than one (e.g., 2, 3, 4, 5, 6, 7, 8, 9, or 10) DNA modification. For example, the translated region and a regulatory element of a genomic locus may each comprise a targeted DNA modification. In certain embodiments, more than one genomic locus of the plant may comprise a DNA modification.

The DNA modification of the genomic locus may be done using any genome modification technique known in the art or described herein. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpfl endonuclease systems, and the like. In some embodiments, the introduction of a DSB can be combined with the introduction of a polynucleotide modification template.

The polynucleotides or recombinant DNA constructs disclosed herein may be used for transformation of any plant species, including, but not limited to, monocots and dicots. Additionally, the genetic modifications described herein may be used to modify any plant species, including, but not limited to, monocots and dicots.

In specific embodiments, plants of the present disclosure are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include, for example, grain plants that provide seeds of interest, oil-seed plants, and leguminous plants. Seeds of interest include, for example, grain seeds, such as corn, wheat, barley, rice, sorghum, rye, etc. Oil-seed plants include, for example, cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea.

For example, in certain embodiments, maize plants are provided that comprise, in their genome, a recombinant DNA construct comprising a polynucleotide that encodes a polypeptide comprising an amino acid sequence that is at least 80% to about 100% identical to any one of SEQ ID NOS: 1-11, 23-31, 40-299, 563, 565, and 567-573. In certain embodiments, the polypeptide comprising an amino acid sequence that is at least 80% to about 100% identical to the amino acid sequence of any one of SEQ ID NOS: 1-11, 23-31, 40-299, 563, 565, and 567-573 comprises the amino acid sequence set forth in SEQ ID NO: 28. In certain embodiments, the polypeptide comprising an amino acid sequence that is at least 80% to about 100% identical to the amino acid sequence of any one of SEQ ID NOS: 1-11, 23-31, 40-299, 563, 565, and 567-573.

In other embodiments, maize plants are provided that comprise a genetic modification at a genomic locus that encodes a polypeptide comprising an amino acid sequence that is at least 80% identical to the amino acid sequence of any one of SEQ ID NOS: 1-11, 23-31, 40-299, 563, 565, and 567-573. In certain embodiments, the polypeptide comprising an amino acid sequence that is at least 80% identical to the amino acid sequence of any one of SEQ ID NOS: 1-11, 23-31, 40-299, 563, 565, and 567-573 comprises the amino acid sequence set forth in SEQ ID NO: 28. In certain embodiments, the polypeptide comprising an amino acid sequence that is at least 80% identical to the amino acid sequence of any one of SEQ ID NOS: 1-11, 23-31, 40-299, 563, 565, and 567-573.

### D. Stacking Other Traits of Interest

In some embodiments, the polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glufosinate herbicides; herbicides which target the protox enzyme (also referred to as “protox inhibitors”).

The plant, plant cell, plant part, seed, and/or grain having an inventive polynucleotide sequence can also be combined with at least one other trait to produce plants that further comprise a variety of desired trait combinations. For instance, the plant, plant cell, plant part, seed, and/or grain having an inventive polynucleotide sequence may be stacked with polynucleotides encoding polypeptides having pesticidal and/or insecticidal activity, or a plant, plant cell, plant part, seed, and/or grain having an inventive polynucleotide sequence may be combined with a plant disease resistance gene.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference. Any plant having an inventive polynucleotide sequence disclosed herein can be used to make a food or a feed product. Such methods comprise obtaining a plant, explant, seed, plant cell, or cell comprising the polynucleotide sequence and processing the plant, explant, seed, plant cell, or cell to produce a food or feed product.

### II. Methods of Use

### A. Methods for Increasing Yield, and/or Increasing the Activity of Polynucleotides in a Plant

Provided are methods for increasing yield in a plant, modifying flowering time of a plant, and/or increasing the activity of one or more polynucleotides disclosed herein in a plant comprising introducing into a plant, plant cell, plant part, seed, and/or grain a recombinant DNA construct comprising any of the inventive polynucleotides described herein, whereby the polypeptide is expressed in the plant. Also provided are methods for increasing yield in a plant, modifying flowering time of a plant, and/or increasing the activity in a plant comprising introducing a genetic modification at a genomic locus of a plant that encodes a polypeptide comprising an amino acid sequence that is at least 80%-99% or 100% identical to the amino acid sequence set for in any one of SEQ ID NOS: 1-11, 23-31, 40-299, 563, 565, and 567-573.

The plant for use in the inventive methods can be any plant species described herein. In certain embodiments, the plant is a grain plant, an oil-seed plant, or leguminous plant. In certain embodiments, the plant is a grain plant such as maize.

As used herein, “yield” refers to the amount of agricultural production harvested per unit of land and may include reference to bushels per acre of a crop at harvest, as adjusted for grain moisture (e.g., typically 15% for maize). Grain moisture is measured in the grain at harvest. The adjusted test weight of grain is determined to be the weight in pounds per bushel, adjusted for grain moisture level at harvest.

In certain embodiments yield is measured in plants grown under optimal growth conditions. As used herein, “optimal conditions” refers to plants that are grown under well-watered or non-drought conditions. In certain embodiments, optimal growth conditions are determined based on the yield of the wild-type control plants in the experiment. As used herein, plants are considered to be grown under optimal conditions when the wild-type plant provides at least 75% of the predicted grain yield.

As used herein, “modifying flowering time” refers to a change in the number of days or growth heat units required for a plant to flower. In certain embodiments, the flowering time of the plant is delayed upon increased expression of the polypeptide. Also contemplated are embodiments in which flowering time is decreased (i.e., less days or growth heat units required for a plant to flower) upon decreased expression of the polypeptide.

As used herein, increase in photosynthetic activity, refers to any detectable increase in the functional activity of the protein compared to a suitable control. The functional activity may be any known biological property of one or more of the polypeptides disclosed herein and includes, for example, increased formation of protein complexes, modulation of biochemical pathways, and/or increased grain yield.

Various methods can be used to introduce a sequence of interest into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

“Stable transformation” is intended to mean that the polynucleotide introduced into a plant integrates into the genome of the plant of interest and is capable of being inherited by the progeny thereof. “Transient transformation” is intended to mean that a polynucleotide is introduced into the plant of interest and does not integrate into the genome of the plant or organism or a polypeptide is introduced into a plant or organism.

Transformation protocols as well as protocols for introducing polypeptides or polynucleotide sequences into plants may vary depending on the type of plant or plant cell, i.e., monocot or dicot, targeted for transformation.

In specific embodiments, the polynucleotide sequences disclosed herein can be provided to a plant using a variety of transient transformation methods. Such transient transformation methods include, but are not limited to, the introduction of the encoded protein directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Various methods can be used to introduce a genetic modification at a genomic locus that encodes and polypeptide into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpfl endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

These fusions contain dCas9 or Cas9 nickase and a suitable deaminase, and they can convert e.g., cytosine to uracil without inducing double-strand break of the target DNA. Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C⋅G to T⋅A conversion and A⋅T to G⋅C conversion at one more locations made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an guidepolynucleotide/Cas endonuclease complex to a unique DNA target site.

The terms “knock-out”, “gene knock-out” and “genetic knock-out” are used interchangeably herein. A knock-out represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

The following are examples of specific embodiments of some aspects of the invention. The examples are offered for illustrative purposes only and are not intended to limit the scope of the invention in any way.

### Example 1

### Protein-Protein Interactions with ZMM28

This example demonstrates the interaction of other polypeptides with Zmm28 transcription factor (SEQ ID NO: 562). MADS-box transcription factors associate as homo- or hetero-dimers to bind CArG box elements and subsequently modulate target gene expression. To identify protein-protein interaction partners that potentially interact with native ZMM28 protein, Yeast Two-Hybrid (Y2H) screening was performed with a B73 immature ear library resulting in the identification of six potential MADS box protein-protein interaction partners (Table 2a).

Since native zmm28 does not express at early growth stages, protein-protein interaction partners contributing to the transgenic maize events phenotypes in seedlings and young leaves were assayed using Y2H screening of a PH184C seedling (V2-V3) library and a B73 V3-V7 leaf library. Nine total interacting proteins, none of which are MADS box proteins, were identified from the two libraries.

Potential interaction partners of ZMM28 were further tested in vivo with a bimolecular fluorescence complementation (BiFC) assay. Following transfection of maize protoplasts, fluorescence was measured indicating interaction between nGFP-Prey and cGFP-ZMM28 (Bait). As BiFC is prone to false-positive self-assembly independent of protein-protein interaction, flow cytometry was used to quantify the BiFC signal and reduce the occurrence of false positives. All signal comparisons were made to a negative control providing a baseline for self-assembly. The control was created by deleting 47 amino acids from the leucine zipper-like K-domain of ZMMADSL6, a protein interaction partner of ZMM28 identified from bioinformatics prediction and Y2H experiment. Truncated ZMMADSL6 (ZMMADSL6-MUT) had significantly reduced interaction with ZMM28 relative to WT ZMMADSL6 while still maintaining nuclear localization. Of the 12 tested protein interactions, eight were confirmed via the BiFC assay with almost half the interactions confirmed positive in both BiFC and Y2H assays (Table 2a). Table 2: ZMM28 protein-protein interactions, transcription, direct targets.

TABLE 2a
Transgenic protein-protein interaction partners.

| ID | Description | Clade | Expression | Y2H | BiFC | HY1H | Sum |
|---|---|---|---|---|---|---|---|
| Zm00001d041781 | ZmZAG2 | AG | 0.001 | + | − | − | + |
| Zm00001d017614 | ZmMADS6 | AGL6 | 0.002 | + | + | + | +++ |
| Zm00001d018667 | ZmZAPL | AP1-FULL | 0.001 | + | + | + | +++ |
| Zm00001d022088 | ZMM28 | AP1 FULL | 1.000 | − | + | − | + |
| Zm00001d028217 | ZmM5 | SEP | 0.005 | + | + | − | ++ |
| Zm00001d031620 | ZmMADSL6 | SEP | 0.007 | + | + | + | +++ |
| Zm00001d021057 | ZmMADS7-LIKE | SEP | 0.002 | + | − | − | + |
| Zm00001d034047 | ZmMADS24 | SEP | 0.007 | − | + | − | + |
| Zm00001d044899 | ZmMADS47-LIKE | SVP | 0.240 | − | − | + | + |
| Zm00001d027957 | ZmM47 | SVP | 1.862 | − | + | + | ++ |
| Zm00001d037925 | ZmSF2 | N/A | 0.534 | + | − | + | ++ |
| Zm00001d022164 | ZmSFT-LIKE | N/A | 0.788 | + | + | + | +++ |

TABLE 2b
Gene ontology enrichment from RNA-seq data

| | | | DEG V6 | Number | | |
|---|---|---|---|---|---|---|
| GO terms | Ontology¹ | Description | Leaf² | in Ref.³ | p-value⁴ | FDR⁵ |
| GO:0015979 | P | photosynthesis | 12 | 94 | 3.10E−10 | 1.40E−07 |
| GO:0009765 | P | photosynthesis, light harvesting | 7 | 25 | 4.50E−09 | 1.10E−06 |
| GO:0019684 | P | photosynthesis, light reaction | 7 | 40 | 1.50E−07 | 2.40E−05 |
| GO:0033013 | P | tetrapyrrole metabolic process | 5 | 39 | 4.50E−05 | 0.0042 |
| GO:0034357 | C | photosynthetic membrane | 7 | 57 | 1.90E−06 | 7.20E−05 |
| GO:0044436 | C | thylakoid part | 6 | 34 | 1.10E−06 | 7.20E−05 |
| GO:0009579 | C | thylakoid | 7 | 60 | 2.70E−06 | 7.20E−05 |
| GO:0004222 | F | metalloendopeptidase activity | 5 | 48 | 0.00012 | 0.038 |
| GO:0006091 | P | generation of precursor metabolites | 14 | 315 | 6.70E−06 | 0.00078 |
| | | and energy | | | | |
| GO:0005975 | P | carbohydrate metabolic process | 20 | 788 | 0.00028 | 0.022 |
| GO:0018130 | P | heterocycle biosynthetic process | 6 | 95 | 0.00043 | 0.028 |

TABLE 2d
Summary results demonstrating ZMM28 interaction with direct target promoters.

| | | Pathway | | Bound in | Log₂ |
|---|---|---|---|---|---|
| ID | Description | (Discovery) | CArG | (assay) | Expression [symbol] |
| Zm00001d053787 | lhca1l, photosystem I light | photosynthesis, | 3 | Protoplast | 0.51* |
| | harvesting complex | light harvesting | | | |
| | gene 1-like | (RNAseq) | | | |
| Zm00001d005814 | lhca5l, photosystem I light | photosynthesis, | 3 | Protoplast | 0.28* |
| | harvesting complex | light harvesting | | | |
| | gene 6-like | (RNAseq) | | | |
| Zm00001d027422 | Photosystem II PsbP, oxygen | Photosynthesis | 3 | Protoplast | 0.24* |
| | evolving complex member | (RNAseq) | | | |
| | (ps2oe) | | | | |
| Zm00001d007267 | lhcb5, light-harvesting | photosynthesis, | 3 | HY1H | 0.22* |
| | complex II chlorophyll a/b | light harvesting | | | |
| | binding protein S | (RNAseq) | | | |
| Zm00001d027694 | Solanesyl diphosphate | Photosynthesis | 3 | Protoplast | 0.2* |
| | synthase 2 chloroplastic | (RNAseq) | | | |
| | (sds) | | | | |
| Zm00001d038163 | Pyruvate, phosphate | Photosynthesis/ | 2 | Protoplast | 0.04 |
| | dikinase | pyruvate | | | |
| | (chloroplastic/cytoplasmic) | metabolism | | | |
| | (ppdk) | (RNAseq) | | | |
| Zm00001d016973 | GID2-like F-box protein | hormone signaling | 2 | HY1H | 0.12 |
| | (gid2) | (ChIPseq) | | | |
| Zm00001d003911 | AFB2-like/TIR1-like (ofb2) | hormone signaling | 5 | HY1H | 0.00 |
| | | (ChIPseq) | | | |
| Zm00001d030995 | b2IP111, CAMP-response | transcription | 5 | Protoplast | 0.23 |
| | element binding protein- | factor activity | | | |
| | related [symbol] | (ChIPseq) | | | |

[symbol]

indicates data missing or illegible when filed

Table 2(a) provides a summary of protein-protein interactions with potential contribution to transgenically expressed zmm28. Expression values are from RNA-seq from transgenic V6 maize leaves and are normalized to zmm28. A “+” was listed for protein-interaction predictions based on yeast two-hybrid (Y2H); maize protoplast BiFC; and in heterodimer yeast one-hybrid (HY1H). (b) GO-term enrichment for transcriptomic analysis of DP202216 V6 leaf tissue. Photosynthesis related includes GO Terms 0015979, 0009765, 0019684, 0006091, 0033013, 0034357, 0044436, and 0009579. (d) Summary of promoter direct target analysis and expression in V6 leaves of event DP202216. 1 P=biological process, F=molecular function, C=cellular component. ² Number of genes associated with each GO term that are differentially expressed between control and event DP202216 V6 leaf, DEG=differentially expressed gene. ³ Total number of genes in each GO category expressed in V6 leaf total detected transcripts. ⁴ Fisher's exact test for GO term enrichment. ⁵ False discovery rate. ⁶ V6 Leaf. * Statistically significant (adjusted p<0.05).

TABLE 2(c)
Differential Expression Values for Genes Involved

log2 fold
change

Photosynthesis related

| | |
|---|---|
| lhcb9 | 0.7188 |
| lhca1l | 0.5080 |
| GRMZM2G436986 | 0.3699 |
| GRMZM2G005433 | 0.3474 |
| GRMZM2G083016 | 0.3327 |
| lhac6l | 0.3099 |
| GRMZM2G059083 | 0.2878 |
| psbs1 | 0.2849 |
| GRMZM2G103101 | 0.2754 |
| GRMZM2G117412 | 0.2752 |
| chlh1 | 0.2710 |
| fdx2 | 0.2506 |
| GRMZM2G089136 | 0.2431 |
| ps2oe | 0.2386 |
| GRMZM2G033885 | 0.2353 |
| lhcb5 | 0.2200 |
| GRMZM2G016066 | 0.2139 |
| sds | 0.2042 |
| ris2 | 0.2018 |
| GRMZM2G064302 | 0.1902 |
| GRMZM2G168143 | 0.1652 |
| GRMZM2G023528 | 0.1622 |
| GRMZM2G027955 | 0.1579 |
| GRMZM2G113325 | 0.1219 |
| GRMZM2G127421 | −0.2992 |
| GRMZM2G359127 | −0.3116 |

Carbohydrate metabolic process

| | |
|---|---|
| GRMZM2G017186 | 0.6168 |
| GRMZM2G083016 | 0.2431 |
| GRMZM2G026807 | 0.2701 |
| GRMZM2G121128 | 0.2688 |
| gbss1b | 0.2450 |
| GRMZM2G089136 | 0.2431 |
| GRMZM2G306732 | 0.2312 |
| GRMZM2G064302 | 0.1902 |
| sps2 | 0.1834 |
| GRMZM2G023528 | 0.1622 |
| GRMZM2G125977 | 0.1599 |
| GRMZM2G027955 | 0.1579 |
| pgm2 | 0.1495 |
| GRMZM2G052546 | 0.1399 |
| mdh6 | 0.0968 |
| GRMZM2G005493 | −0.1821 |
| umc2230 | −0.2250 |
| GRMZM2G122431 | −0.6249 |
| GRMZM2G082034 | −0.8355 |
| GRMZM2G347708 | −0.9058 |

Heterocycle biosynthetic process

| | |
|---|---|
| thi1 | 0.3236 |
| GRMZM2G177412 | 0.3099 |
| chlh1 | 0.2710 |
| GRMZM2G027663 | 0.2200 |
| GRMZM2G023528 | 0.1622 |
| GRMZM2G113325 | 0.1219 |

Metalloendopeptidase activity

| | |
|---|---|
| GRMZM2G044697 | 0.2005 |
| GRMZM2G087598 | 0.1873 |
| prep2 | 0.1714 |
| GRMZM2G111200 | 0.1703 |
| GRMZM2G163193 | 0.1281 |

Pathway analysis of differentially expressed gene transcripts are shown above. Log 2 fold change heat maps of differentially expressed genes functioning in enriched pathways in event DP202216 V6 leaf tissue.

### Example 2

### Identification of Direct Interacting Partners

Yeast two-hybrid assay. A commercially available yeast two-hybrid system (Clontech (USA)/Takara (Japan) was used to discover and test for potential protein interaction partners with ZMM28. Three maize cDNA prey libraries were constructed from B73 V12-V14 immature ear, PH184C V2-V3 whole seedling, and B73 V3-V7 leaf RNA. The cDNA libraries were generated using SMART technology and co-transformed with linearized pGADT7-Rec into Yeast Strain Y187. At least one million prey clones from each library were mated to a ZMM28 bait strain. Mating was continued until zygotes could be observed using a light microscope and then plated on QDO/-Ade/-His/-Leu/-Trp and incubated at 30° C. for 5 days. Identified protein interaction partners were re-transformed into the Y2H system for confirmation testing.

Bimolecular fluorescence complementation (BiFC). Coding sequences for candidate protein-protein interaction partners to ZMM28 were synthesized by GenScript (USA) and placed under the control of the ZmGos2 promoter with a ZmUbi intron 1. The coding sequences were translationally fused to the C-terminal or N-terminal part of the monomeric Ac-GFP1 (Clontech, USA/Takara, Japan) with a 30× Glutamine linker. ZMMADSL6 was selected as a positive control in the BiFC assay as it was confirmed to interact with ZMM28 by Y2H. A truncated version of ZMMADSL6 without the protein interaction domain (a leucine zipper like region in the K-domain) was generated as a negative control.

Maize seedlings were germinated and grown in Fafard Super Fine Germination Mix for 6 days in a lighted growth chamber (30° C., 60% RH, 24 h light) and were transferred to a dark growth chamber (30° C., 60% RH, 0 h light) and grown for an additional 4 days to V1. Seedlings were sub-irrigated with deionized water. Maize protoplasts were isolated from these seedlings and were transiently transformed by PEG-mediated transfection as described by Yoo et al.63 with the addition of 0.6 M mannitol in the enzyme, WI, W5 and MMG solutions. Protoplasts were transfected with 10 pmol bait+10 pmol prey plasmid DNA per 3×104 cells. Protoplasts were incubated on a 12-well (1 mL WI) plate for 20 hours at RT before samples were analyzed.

BiFC signals were detected by flow cytometry performed using an Attune™ Flow Cytometer (Thermo Fisher Scientific, Waltham, Mass., USA) with a Blue/Violet configuration (488 nm, 20 mW laser and a 405 nm, 50 mW laser). Protoplasts were first gated (R1) by forward scatter (FSC) and side scatter (SCC) to identify 10,000 intact cells (events) and subsequently analyzed for fluorescence emission measured on BL1 (530/30 nm band pass filter) and BL2 (574/26 nm band pass filter) to distinguish between cells exhibiting the BiFC signal (R2) and auto-fluorescence. At least two independent experiments were performed for each protein interaction test with the positive and negative controls present in every experiment. All experiments were designed and analyzed as single factor randomized complete blocks with n=4. Significant differences were determined by analysis of variance with P<0.05 comparing protein interaction partners to the truncated ZMMADSL6 (ZMMADSL6-MUT) negative control. Western blots were used to confirm expression in cells transfected with the negative control ZMMADSL6-MUT (prey) and ZMM28 (bait).

RNA-Seq of ZmGos2-zmm28 and control and data analysis. DP202216 was selected for in-depth molecular analysis due to its more favorable insertion region. RNA-Seq libraries were constructed from four biological replicates of control and DP202216 youngest fully expanded leaves at V6 stage. Sequencing was performed on an Illumina HiSeq2500 (Illumina, Inc., USA) with a total read count of 154 million, and a minimum of 12 million reads per sample. RNA-Seq data were aligned to a proprietary maize B73 reference genome using Bowtie 2. Overall loci abundances were estimated using the expected fragment counts metric computed by RSEM65. Samples were vetted for quality by “Robust Principal Components based on Projection Pursuit (PP): GRID search Algorithm” in the “Scalable Robust Estimators with High Breakdown Point” R package [https://cran.r-project.org/package=rrcov]. Fold change was computed and hypothesis tests for differential expression were run using DESeq2, which fits the following model:
Kᵢⱼ˜NB (pᵢⱼ, αᵢ)=
μᵢⱼ=sⱼgᵢⱼ
log₂ qᵢⱼ=x_{j.}βₜ

Where Kᵢⱼ is the observed count for gene i in sample j following a Negative Binomial distribution, (μᵢⱼ, αᵢ, sⱼ, qᵢⱼ) are all parameters fit to the data (see citation), x_{j.} is 1 if sample j is transgenic and 0 if it is control and βᵢ contains the log₂ fold changes for gene i across all high-nitrogen leaf samples.

All genes in the proprietary reference genome were converted to public gene model identifiers. The DEGs were then annotated with Gene Ontology terms and differential gene set enrichment was done comparing to the total publicly mapped transcript set for the entire V6 leaf data set using AgriGO.

Chromatin immunoprecipitation and sequencing (ChIP-seq) data analysis. Chromatin immunoprecipitation (ChIP) was performed in duplicate on the youngest fully expanded leaf from V4 control and DP202216 maize plants using an anti-ZMM28 antibody (R743); ChIP without antibody was included as a control for each sample. Sequencing was performed on an Illumina HiSeq2500 (Illumina, Inc., USA) with a total read count of 461 million, with a minimum of 26 million reads per sample. ChIP-Seq reads were aligned to a proprietary maize B73 reference genome using Bowtie 2. Alignments were then fed to MACS 2.071 in order to detect differential binding in the transgenic samples. Reproducible peaks were then selected using Irreproducible Discovery Rate (IDR) analysis.

TABLE 3
Maize protoplast Bimolecular Fluorescence Complementation assay summary

| | | | Positive | | | | | |
|---|---|---|---|---|---|---|---|---|
| | | | control cell | | GOI cell | | | |
| | | Negative | count | | count | | | |
| | | control | (Positive vs. | | (GOI vs. | | GOI vs. | |
| | Protein | cell | negative p | | negative p | | positive | |
| GOI ID | description | count | value) | | value) | | (p-value) | Summary |
| Zm00001d041781 | ZmZAG2 | 65 | 155 | (0.027) | 125 | (0.053) | 0.195 | − |
| Zm00001d017614 | ZmMADS6 | 375 | 1589 | (0.002) | 1899 | (0.002) | 0.146 | + |
| Zm00001d018667 | ZmZAPL | 491 | 1538 | (0.037) | 2161 | (0.010) | 0.285 | + |
| Zm00001d022088 | ZMM28 | 483 | 878 | (0.010) | 1014 | (0.034) | 0.429 | + |
| Zm00001d028217 | ZmM5 | 2085 | 4728 | (0.007) | 4512 | (0.004) | 0.675 | + |
| Zm00001d031620 | ZmMADSL6 | 2396 | N/A | (N/A) | 4059 | (0.002) | N/A | + |
| Zm00001d021057 | ZmMADS7- | 421 | 1321 | (0.006) | 438 | (0.427) | 0.010 | − |
| | LIKE | | | | | | | |
| Zm00001d034047 | ZmMADS24 | 586 | 2024 | (0.044) | 2099 | (0.035) | 0.764 | + |
| Zm00001d044899 | ZmMADS47- | 483 | 878 | (0.010) | 254 | (0.047) | 0.006 | − |
| | LIKE | | | | | | | |
| Zm00001d027957 | ZmM47 | 483 | 878 | (0.010) | 1085 | (0.005) | 0.107 | + |
| Zm00001d037925 | ZmSF2 | 483 | 878 | (0.010) | 715 | (0.077) | 0.363 | − |
| Zm00001d022164 | ZmSFT-LIKE | 421 | 1321 | (0.006) | 2328 | (0.016) | 0.016 | + |

Values represent BiFC positive cell counts from selected gate strategies out of 10,000 cells in representative flow cytometry experiments. Negative- and positive-control cell counts represent fluorescent cells in protoplast populations co-transfected with BiFC fusion constructs of truncated ZmMADSL6 and ZMM28 or full length ZmMADSL6 and ZMM28, respectively. GOI=gene of interest. +/− indicates whether the BiFC assay was concluded to be positive (+) or negative (−) based on the p-value calculations.

### Example 3

### Direct-Target Analysis

This example demonstrates the analysis behind identification of the direct targets of Zmm28. To identify genes directly modulated by transgenic ZMM28 and their associated pathways, genomic sequences directly bound by ZMM28 were recovered from leaves of control and DP202216 plants at the V4 stage, at which time no detectable native ZMM28 protein is produced, and analyzed by chromatin immunoprecipitation and sequencing (ChIP-Seq). In addition, putative direct targets of the transgenic ZMM28 were identified from CArG-motif enrichment of the promoters from the strong differentially regulated DEGs in the transcriptome experiment.

Two in-cell assays were used to collectively validate candidate direct target promoters identified from above two experiments. A heterodimer Yeast One-Hybrid (HY1H) assay analyzed the capability of ZMM28 and one of its protein-protein interaction partners to directly bind a promoter. Additionally, V2 etiolated maize protoplast cells were used in a protoplast direct-target assay using a ZsGreen1 reporter to detect ZMM28 interactions with promoters. The HY1H assay provided predetermined heterodimer interaction partners while the maize protoplast direct-target assay potentially tested ZMM28 homodimers or heterodimers, forming between native protein-protein interaction partners. Promoters of key photosynthetic pathway components were bound by ZMM28, as were promoters of gibberellin and auxin receptor genes which are responsible for sensing these phytohormones (Table 2d).

Potential direct target genes (directly bound by the ZMM28 transcription factor) in V4 leaf ChIP-Seq data and select RNA-seq DEG candidates which contain CArG-boxes in the 3 kb upstream of the coding sequence were then screened based on potential function. Promoters of genes with known functional relevance and identifiable CArG sequences were synthesized (Genscript, USA) for direct target assays. Synthesized sequences were cloned into pAbAi (Clontech, USA/TAKARA, Japan) for inclusion in Yeast One-Hybrid assays with ZMM28 and Heterodimer Yeast One-Hybrid assays with ZMM28 and ZMM28 protein-protein interaction partners. Promoter sequences were integrated into the Yeast One-Hybrid Gold strain and individually transformed with a ZMM28-prey plasmid to test for protein-DNA interactions per the Yeast One-Hybrid manual. Heterodimer Yeast One-Hybrid was similarly performed, but including ZMM28 encoded on a Yeast Two-Hybrid bait plasmid (pGBK-T7) and ZMM28 protein-protein interaction partners encoded on a prey (pGAD-T7) plasmid.

Plant cell-based direct target assays were conducted in maize protoplasts. Protoplasts were isolated and transfected as described above. Reporter constructs consisted of the synthesized promoter sequences identified above transcriptionally fused to a ZsGreen1 (Clontech, USA/TAKARA, Japan) coding sequence followed by a pinII terminator. Effector constructs comprised a maize Gos2 promoter followed by a maize ZmUbi intron driving an effector protein coding sequence. Effector proteins were ZMM28, ZMM28 translationally fused to a 5×VP16 transcriptional activation domain, or β-glucuronidase as a negative control. Protoplasts were evaluated with flow cytometry with similar methodology to above.

### Example 4

### Transcriptome Analysis to Identify Differentially Expressed Genes

This example demonstrates identification of several differentially expressed genes in plants expressing Zmm28 transgenically. Transcriptome analysis was conducted to identify differentially-expressed genes (DEGs) and their associated pathways that could provide a possible molecular basis for the previously described increased photosynthesis, N uptake, and plant growth. For simplicity, RNA-seq analysis was focused on V6 leaves from DP202216 and control plants. Results of this analysis identified 192 up-regulated and 64 down-regulated transcripts in DP202216 leaves as compared to the control leaf data (Table 3). CArG box sequences were contained within 3 kb upstream of their promoters in 76% of the DEGs, relative to 26-28% of DEGs from two over-expressed non-MADS transcription factors over a total of four experiments. These results suggest that many of the DEGs may be directly regulated by transgenic ZMM28 binding to their promoters at the V6 stage.

To further gain a global view of the differentially-expressed gene (DEG) function, Gene Ontology (GO) enrichment analysis was conducted and 11 GO terms were identified in the V6 leaf DEG dataset (Table 2b,c). Photosynthesis, generation of precursor metabolites and energy, as well as carbohydrate metabolic processes were the three main GO terms identified, all of which could contribute to promote plant growth and development. These results are consistent with the measured phenotypes of ZmGos2-zmm28 plants and suggest that photosynthesis and carbon assimilation-related genes expression are responsible for the measured grain yield increase.

Polynucleotide sequences encoding a polypeptide represented by one of SEQ ID NOS: 40-222 and polynucleotide sequences represented by one of SEQ ID NOS: 285-484 and 548-561 exhibit increased expression in plants that have increased and extended expression of Zmm28, compared to a control maize plant. Therefore, these sequences and their allelic variants representing about 95% sequence identity to one of SEQ ID NOS: 40-222, 285-484 and 548-561 are suitable for expression modulation and/or activity modulation to improve agronomic characteristics of maize. This can be achieved by a variety of means, transgenic up-regulation, marker-assisted breeding that selects for increased expression alleles, genome editing or genome engineering that employ site-specific DNA modification, screening for naturally occurring variants or induced mutagenized populations or a combination thereof

Polynucleotide sequences encoding a polypeptide represented by one of SEQ ID NOS: 223-284 and polynucleotide sequences represented by one of SEQ ID NOS: 485-547 exhibit reduced expression in plants that have increased and extended expression of Zmm28. Therefore, these sequences and their allelic variants representing about 95% sequence identity to one of SEQ ID NOS: 223-284 and 485-547 are suitable for expression modulation and/or activity modulation to improve agronomic characteristics of maize. This can be achieved by a variety of means, transgenic down-regulation, marker-assisted breeding that selects for reduced expression alleles, genome editing or genome engineering that employ site-specific DNA modification, screening for naturally occurring variants or induced mutagenized populations or a combination thereof.

TABLE 4
List of differentially expressed genes at a 95% confidence
interval in DP202216 vs control V6 leaf tissue

| | Base | log₂ fold | adjusted |
|---|---|---|---|
| ID | mean | change | p-value |
| Zm00001d022088 | 366.7 | 2.161 | 0.000 |
| Zm00001d023455 | 399.2 | 1.309 | 0.000 |
| Zm00001d023456 | 775.4 | 1.175 | 0.000 |
| Zm00001d038273 | 268.1 | 1.056 | 0.000 |
| Zm00001d004053 | 788.2 | 1.054 | 0.000 |
| Zm00001d029183 | 467.1 | 0.885 | 0.018 |
| Zm00001d051194 | 422.1 | 0.735 | 0.006 |
| Zm00001d033132 | 1007.7 | 0.719 | 0.000 |
| Zm00001d029215 | 401.8 | 0.710 | 0.049 |
| Zm00001d033543 | 949.1 | 0.710 | 0.010 |
| Zm00001d053925 | 887.4 | 0.695 | 0.003 |
| Zm00001d028269 | 226.6 | 0.651 | 0.000 |
| Zm00001d033544 | 255.6 | 0.643 | 0.000 |
| Zm00001d034015 | 844.9 | 0.617 | 0.000 |
| Zm00001d027743 | 384.8 | 0.601 | 0.000 |
| Zm00001d048311 | 698.4 | 0.565 | 0.000 |
| Zm00001d053787 | 4263.0 | 0.508 | 0.019 |
| Zm00001d047256 | 331.2 | 0.490 | 0.000 |
| Zm00001d048720 | 258.8 | 0.440 | 0.017 |
| Zm00001d023426 | 2247.4 | 0.436 | 0.004 |
| Zm00001d004331 | 771.7 | 0.430 | 0.049 |
| Zm00001d050748 | 566.4 | 0.424 | 0.000 |
| Zm00001d010321 | 1082.7 | 0.416 | 0.008 |
| Zm00001d004894 | 15128.1 | 0.403 | 0.000 |
| Zm00001d042346 | 506.7 | 0.401 | 0.045 |
| Zm00001d031657 | 1064.8 | 0.393 | 0.002 |
| Zm00001d008178 | 262.0 | 0.377 | 0.042 |
| Zm00001d043044 | 3741.8 | 0.371 | 0.001 |
| Zm00001d018623 | 374.5 | 0.370 | 0.002 |
| Zm00001d043095 | 820.2 | 0.369 | 0.000 |
| Zm00001d029062 | 448.4 | 0.354 | 0.018 |
| Zm00001d011900 | 1851.3 | 0.347 | 0.001 |
| Zm00001d010672 | 3578.8 | 0.333 | 0.003 |
| Zm00001d011183 | 6346.0 | 0.324 | 0.049 |
| Zm00001d037103 | 1178.1 | 0.322 | 0.039 |
| Zm00001d009028 | 28412.8 | 0.318 | 0.038 |
| Zm00001d013937 | 3658.0 | 0.318 | 0.008 |
| Zm00001d002873 | 412.1 | 0.315 | 0.010 |
| Zm00001d037362 | 731.7 | 0.311 | 0.039 |
| Zm00001d026404 | 8690.7 | 0.310 | 0.028 |
| Zm00001d047255 | 2253.8 | 0.309 | 0.024 |
| Zm00001d031253 | 5805.9 | 0.309 | 0.000 |
| Zm00001d027576 | 856.8 | 0.306 | 0.011 |
| Zm00001d052595 | 12058.5 | 0.304 | 0.002 |
| Zm00001d013367 | 1046.5 | 0.301 | 0.007 |
| Zm00001d046001 | 2845.8 | 0.297 | 0.049 |
| Zm00001d008963 | 418.8 | 0.296 | 0.049 |
| Zm00001d048515 | 366.0 | 0.290 | 0.043 |
| Zm00001d018779 | 5833.9 | 0.289 | 0.028 |
| Zm00001d052595 | 5155.7 | 0.289 | 0.020 |
| Zm00001d042049 | 5095.7 | 0.288 | 0.016 |
| Zm00001d040242 | 573.1 | 0.287 | 0.022 |
| Zm00001d042697 | 29924.1 | 0.285 | 0.000 |
| Zm00001d019518 | 2389.6 | 0.283 | 0.004 |
| Zm00001d048313 | 568.8 | 0.283 | 0.034 |
| Zm00001d033150 | 2844.8 | 0.281 | 0.000 |
| Zm00001d007858 | 4471.7 | 0.280 | 0.000 |
| Zm00001d003588 | 1202.7 | 0.280 | 0.005 |
| Zm00001d036903 | 1562.2 | 0.279 | 0.019 |
| Zm00001d031484 | 3091.2 | 0.276 | 0.000 |
| Zm00001d018157 | 2066.8 | 0.275 | 0.012 |
| Zm00001d005814 | 3964.9 | 0.275 | 0.000 |
| Zm00001d033338 | 1889.2 | 0.275 | 0.028 |
| Zm00001d053446 | 1126.3 | 0.272 | 0.027 |
| Zm00001d026603 | 26238.1 | 0.271 | 0.000 |
| Zm00001d027841 | 19250.6 | 0.270 | 0.016 |
| Zm00001d030048 | 4061.0 | 0.269 | 0.001 |
| Zm00001d005346 | 4426.3 | 0.267 | 0.025 |
| Zm00001d023706 | 4737.6 | 0.267 | 0.000 |
| Zm00001d042050 | 13900.2 | 0.266 | 0.013 |
| Zm00001d042533 | 870.8 | 0.262 | 0.019 |
| Zm00001d022590 | 756.8 | 0.261 | 0.024 |
| Zm00001d045431 | 3088.4 | 0.260 | 0.045 |
| Zm00001d047743 | 2167.9 | 0.260 | 0.000 |
| Zm00001d036738 | 4088.9 | 0.251 | 0.000 |
| Zm00001d018274 | 2320.0 | 0.251 | 0.020 |
| Zm00001d035003 | 9178.4 | 0.251 | 0.028 |
| Zm00001d027321 | 906.8 | 0.250 | 0.013 |
| Zm00001d014284 | 2863.7 | 0.249 | 0.000 |
| Zm00001d036340 | 3635.8 | 0.248 | 0.039 |
| Zm00001d003767 | 684.3 | 0.248 | 0.021 |
| Zm00001d031997 | 2770.7 | 0.246 | 0.022 |
| Zm00001d019479 | 7547.5 | 0.245 | 0.000 |
| Zm00001d024148 | 10915.4 | 0.244 | 0.013 |
| Zm00001d038579 | 22011.0 | 0.243 | 0.000 |
| Zm00001d037273 | 2559.2 | 0.240 | 0.028 |
| Zm00001d025845 | 682.1 | 0.239 | 0.033 |
| Zm00001d027422 | 1300.4 | 0.239 | 0.049 |
| Zm00001d024519 | 15382.2 | 0.238 | 0.000 |
| Zm00001d040163 | 4199.3 | 0.238 | 0.000 |
| Zm00001d012868 | 2451.1 | 0.237 | 0.001 |
| Zm00001d021763 | 46089.8 | 0.235 | 0.049 |
| Zm00001d035761 | 2876.1 | 0.232 | 0.028 |
| Zm00001d032301 | 1829.9 | 0.231 | 0.003 |
| Zm00001d028562 | 7597.8 | 0.231 | 0.048 |
| Zm00001d034538 | 1219.9 | 0.231 | 0.042 |
| Zm00001d048116 | 1310.7 | 0.230 | 0.003 |
| Zm00001d048998 | 33243.0 | 0.226 | 0.003 |
| Zm00001d049490 | 896.9 | 0.225 | 0.049 |
| Zm00001d015975 | 1389.6 | 0.224 | 0.030 |
| Zm00001d021435 | 101585.8 | 0.223 | 0.001 |
| Zm00001d015613 | 1517.4 | 0.221 | 0.049 |
| Zm00001d039258 | 45911.3 | 0.221 | 0.023 |
| Zm00001d007267 | 22445.6 | 0.220 | 0.000 |
| Zm00001d033383 | 8820.9 | 0.220 | 0.040 |
| Zm00001d026645 | 774.2 | 0.220 | 0.028 |
| Zm00001d023757 | 3788.0 | 0.219 | 0.026 |
| Zm00001d034005 | 1328.0 | 0.218 | 0.017 |
| Zm00001d022381 | 1437.7 | 0.215 | 0.028 |
| Zm00001d031962 | 1481.3 | 0.214 | 0.012 |
| Zm00001d005446 | 17349.9 | 0.214 | 0.017 |
| Zm00001d027511 | 1065.8 | 0.211 | 0.034 |
| Zm00001d017178 | 1469.3 | 0.210 | 0.020 |
| Zm00001d038947 | 2902.2 | 0.209 | 0.007 |
| Zm00001d009982 | 2640.8 | 0.209 | 0.006 |
| Zm00001d034739 | 1282.4 | 0.208 | 0.033 |
| Zm00001d044745 | 2078.9 | 0.208 | 0.002 |
| Zm00001d038491 | 1871.3 | 0.208 | 0.007 |
| Zm00001d014445 | 2012.3 | 0.207 | 0.029 |
| Zm00001d039745 | 2541.4 | 0.207 | 0.049 |
| Zm00001d038485 | 2954.2 | 0.207 | 0.020 |
| Zm00001d012287 | 3196.9 | 0.205 | 0.042 |
| Zm00001d027694 | 4079.0 | 0.204 | 0.002 |
| Zm00001d003470 | 2149.7 | 0.204 | 0.049 |
| Zm00001d019180 | 1691.1 | 0.203 | 0.001 |
| Zm00001d007394 | 2809.7 | 0.203 | 0.046 |
| Zm00001d053432 | 14717.4 | 0.202 | 0.000 |
| Zm00001d039900 | 3507.8 | 0.200 | 0.004 |
| Zm00001d050810 | 2267.9 | 0.200 | 0.013 |
| Zm00001d016802 | 3694.7 | 0.200 | 0.024 |
| Zm00001d004978 | 1355.2 | 0.199 | 0.049 |
| Zm00001d028924 | 2314.7 | 0.199 | 0.017 |
| Zm00001d002815 | 6335.2 | 0.198 | 0.034 |
| Zm00001d029065 | 1335.9 | 0.198 | 0.033 |
| Zm00001d052184 | 1429.5 | 0.197 | 0.005 |
| Zm00001d038337 | 1709.0 | 0.193 | 0.049 |
| Zm00001d003713 | 843.8 | 0.193 | 0.049 |
| Zm00001d031953 | 2062.3 | 0.193 | 0.049 |
| Zm00001d053576 | 1070.5 | 0.191 | 0.036 |
| Zm00001d000123 | 4446.2 | 0.191 | 0.010 |
| Zm00001d045431 | 12598.3 | 0.190 | 0.039 |
| Zm00001d036630 | 26955.9 | 0.187 | 0.000 |
| Zm00001d048515 | 4138.4 | 0.186 | 0.044 |
| Zm00001d021310 | 10622.2 | 0.185 | 0.009 |
| Zm00001d038037 | 13570.3 | 0.184 | 0.005 |
| Zm00001d042353 | 1578.4 | 0.183 | 0.029 |
| Zm00001d050150 | 1799.2 | 0.183 | 0.014 |
| Zm00001d025545 | 10158.4 | 0.182 | 0.000 |
| Zm00001d012168 | 1918.6 | 0.182 | 0.006 |
| Zm00001d011581 | 2421.3 | 0.176 | 0.013 |
| Zm00001d018030 | 12550.6 | 0.173 | 0.012 |
| Zm00001d018145 | 15856.7 | 0.171 | 0.012 |
| Zm00001d040221 | 4822.7 | 0.170 | 0.001 |
| Zm00001d033594 | 4970.0 | 0.170 | 0.006 |
| Zm00001d008625 | 3404.2 | 0.170 | 0.015 |
| Zm00001d032380 | 6439.8 | 0.169 | 0.042 |
| Zm00001d045575 | 9388.9 | 0.165 | 0.012 |
| Zm00001d042526 | 12885.2 | 0.164 | 0.024 |
| Zm00001d043168 | 9130.5 | 0.164 | 0.014 |
| Zm00001d053545 | 13321.4 | 0.162 | 0.012 |
| Zm00001d053981 | 2247.8 | 0.162 | 0.031 |
| Zm00001d017746 | 3272.3 | 0.162 | 0.023 |
| Zm00001d012083 | 3531.8 | 0.161 | 0.002 |
| Zm00001d024718 | 1276.1 | 0.160 | 0.039 |
| Zm00001d021621 | 29252.0 | 0.160 | 0.033 |
| Zm00001d015004 | 3642.0 | 0.159 | 0.031 |
| Zm00001d039131 | 6755.2 | 0.158 | 0.013 |
| Zm00001d044970 | 11313.1 | 0.158 | 0.015 |
| Zm00001d018401 | 3854.9 | 0.156 | 0.007 |
| Zm00001d015975 | 2687.4 | 0.150 | 0.024 |
| Zm00001d013428 | 6916.2 | 0.150 | 0.002 |
| Zm00001d021246 | 8907.5 | 0.148 | 0.041 |
| Zm00001d007921 | 1700.3 | 0.146 | 0.045 |
| Zm00001d027511 | 11194.0 | 0.144 | 0.039 |
| Zm00001d027309 | 2776.5 | 0.140 | 0.031 |
| Zm00001d025544 | 4067.6 | 0.138 | 0.040 |
| Zm00001d039276 | 2421.5 | 0.137 | 0.049 |
| Zm00001d003512 | 7940.7 | 0.134 | 0.016 |
| Zm00001d053861 | 4328.4 | 0.130 | 0.048 |
| Zm00001d038894 | 18625.0 | 0.129 | 0.007 |
| Zm00001d051321 | 5842.9 | 0.128 | 0.039 |
| Zm00001d016826 | 6846.9 | 0.126 | 0.031 |
| Zm00001d016854 | 3875.9 | 0.122 | 0.032 |
| Zm00001d017435 | 4798.8 | 0.121 | 0.018 |
| Zm00001d048373 | 16010.1 | 0.109 | 0.019 |
| Zm00001d018901 | 18599.9 | 0.104 | 0.043 |
| Zm00001d019454 | 18340.3 | 0.100 | 0.046 |
| Zm00001d031899 | 38207.2 | 0.097 | 0.036 |
| Zm00001d045706 | 13660.8 | −0.114 | 0.003 |
| Zm00001d035869 | 3703.4 | −0.169 | 0.035 |
| Zm00001d053262 | 2715.0 | −0.177 | 0.034 |
| Zm00001d017958 | 1986.1 | −0.179 | 0.042 |
| Zm00001d007113 | 2081.6 | −0.182 | 0.007 |
| Zm00001d051650 | 1133.1 | −0.205 | 0.025 |
| Zm00001d015138 | 60518.8 | −0.222 | 0.000 |
| Zm00001d030103 | 1549.1 | −0.225 | 0.034 |
| Zm00001d021846 | 1819.7 | −0.226 | 0.000 |
| Zm00001d041576 | 849.3 | −0.252 | 0.039 |
| Zm00001d005391 | 618.2 | −0.255 | 0.019 |
| Zm00001d009022 | 4240.0 | −0.264 | 0.023 |
| Zm00001d041343 | 1309.4 | −0.268 | 0.045 |
| Zm00001d039965 | 528.6 | −0.274 | 0.025 |
| Zm00001d029047 | 509.7 | −0.275 | 0.021 |
| Zm00001d028230 | 2507.9 | −0.284 | 0.012 |
| Zm00001d013243 | 725.6 | −0.286 | 0.040 |
| Zm00001d032926 | 458.9 | −0.299 | 0.034 |
| Zm00001d045667 | 1861.6 | −0.302 | 0.000 |
| Zm00001d021846 | 701.7 | −0.303 | 0.025 |
| Zm00001d033469 | 934.7 | −0.312 | 0.032 |
| Zm00001d019563 | 3360.0 | −0.314 | 0.007 |
| Zm00001d003866 | 945.1 | −0.316 | 0.008 |
| Zm00001d039325 | 1802.7 | −0.321 | 0.000 |
| Zm00001d023516 | 69804.9 | −0.324 | 0.036 |
| Zm00001d050918 | 301.0 | −0.325 | 0.029 |
| Zm00001d025253 | 331.5 | −0.346 | 0.040 |
| Zm00001d024499 | 1459.3 | −0.364 | 0.029 |
| Zm00001d045948 | 478.6 | −0.374 | 0.013 |
| Zm00001d021569 | 473.1 | −0.376 | 0.024 |
| Zm00001d053327 | 601.3 | −0.377 | 0.002 |
| Zm00001d047208 | 736.1 | −0.379 | 0.000 |
| Zm00001d007687 | 1551.8 | −0.385 | 0.001 |
| Zm00001d015126 | 1333.7 | −0.395 | 0.005 |
| Zm00001d034781 | 288.2 | −0.403 | 0.006 |
| Zm00001d016655 | 1486.6 | −0.412 | 0.049 |
| Zm00001d043517 | 322.3 | −0.426 | 0.013 |
| Zm00001d045667 | 288.4 | −0.427 | 0.049 |

Base Mean = the mean of counts of all samples, normalized for sequencing depth.

### Example 5

### Transcriptome Analysis to Identify Differentially Expressed Genes

This example demonstrates identification of several differentially expressed genes in plants expressing Zmm28 transgenically. Transcriptome analysis was conducted to identify differentially-expressed genes (DEGs) and their associated pathways that could provide a possible molecular basis for the previously described increased photosynthesis, N uptake, and plant growth. For simplicity, RNA-seq analysis was focused on V6 leaves from DP202216 and control plants. Results of this analysis identified 192 up-regulated and 64 down-regulated transcripts in DP202216 leaves as compared to the control leaf data (Table 4). CArG box sequences were contained within 3 kb upstream of their promoters in 76% of the DEGs, relative to 26-28% of DEGs from two over-expressed non-MADS transcription factors over a total of four experiments.

### Example 6

### Increasing Photosynthetic Flux Through Gene Expression Alteration of One or More Sequences

In some embodiments, altering expression levels of direct target genes or their protein products/activities thereof (e.g., polypeptides represented by SEQ ID NOS: 23-31 or polynucleotides represented by SEQ ID NOS: 32-39)) may positively affect grain yield. In particular embodiments, altering expression or gene product amounts may change photosynthetic flux. For example, increasing expression of polypeptides represented by SEQ ID NOS: 23-31 may lead to increases in photosynthesis as measured by increased CO₂ exchange rate (CER) or electron transport rate. In other examples, expression changes may lead to positive feedback resulting in both increased leaf area with increased light interception and increased photosynthetic rate per leaf area. In yet other examples, expression changes in polypeptides represented by SEQ ID NOS: 23-31 may lead to increased photosynthate and enhanced uptake and assimilation of nitrogen. Increases or decreases in gene expression levels is achieved by introducing a targeted genetic modification or through expression of a recombinant DNA construct. Targeted genetic modifications include for example, removing a repressor element from the regulatory region of the gene or by mutating one or more motifs to increase expression levels of the gene. Enhancer elements can also be introduced to increase gene expression.

### Example 6

### Increasing Plant Growth and/or N-Assimilation Through Gene Expression Alteration

In some embodiments, altering gene expression of direct target genes may enhance plant growth. In particular embodiments, altering gene expression may lead to enhanced early vigor or enhanced grain yield. In some embodiments, changes in expression of SEQ ID NO: 30, 38, 563 and/or 564 lead to enhancement of phytohormone reception response leading to increased plant growth or enhanced grain yield.

In some embodiments, altering gene expression of direct target genes may enhance nitrogen uptake or assimilation. In particular embodiments, changes in expression of SEQ ID NO: 39 or related genes such as SEQ ID NO: 566 lead to improvements in nitrogen uptake or assimilation. Such improvements may further lead to enhanced plant growth and/or grain yield.

Increases or decreases in gene expression levels is achieved by introducing a targeted genetic modification or through expression of a recombinant DNA construct. Targeted genetic modifications include for example, removing a repressor element from the regulatory region of the gene or by mutating one or more motifs to increase expression levels of the gene. Enhancer elements can also be introduced to increase gene expression

### Example 7

### Increasing Plant Stress Tolerance

In some embodiments, altering gene expression of direct target genes may enhance plant stress or disease tolerance. In particular embodiments, changes in expression of SEQ ID NOS: 574-579 results in improvements in biotic or abiotic stress tolerance. In particular embodiments, such expression changes and stress tolerance may lead to enhanced plant vigor, enhanced biomass accumulation and/or enhanced grain yield.

Increases or decreases in gene expression levels to improve plant stress tolerance is achieved by introducing a targeted genetic modification or through expression of a recombinant DNA construct. Targeted genetic modifications include for example, removing a repressor element from the regulatory region of the gene or by mutating one or more motifs to increase expression levels of the gene. Enhancer elements can also be introduced to increase gene expression

Terms used in the claims and specification are defined as set forth below unless otherwise specified. It must be noted that, as used in the specification and the appended claims, the singular forms “a,” “an” and “the” include plural referents unless the context clearly dictates otherwise.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1-31. (canceled)

32. A method for increasing grain or seed or biomass yield in a plant, the method comprising:
a. expressing in a regenerable plant cell a recombinant DNA construct comprising a regulatory element operably linked to a polynucleotide encoding a polypeptide comprising an amino acid sequence that is at least 80% identical to an amino acid sequence selected from the group consisting of SEQ ID NOS: 1-11, 23-31, 40-299, 563, 565, and 567-573; and
b. generating the plant, wherein the plant comprises in its genome the recombinant DNA construct.

33. The method of claim 32, wherein the regulatory element is a heterologous promoter.

34. The method of claim 32, wherein the polypeptide is at least 90% identical to an amino acid sequence selected from the group consisting of SEQ ID NOS: 1-11, 23-31, 40-299, 563, 565, and 567-573.

35. The method of claim 32, wherein the polynucleotide encodes a polypeptide comprising an amino acid sequence that is at least 95% identical to a full length amino acid sequence selected from the group consisting of SEQ ID NOS: 1-11, 23-31, 40-299, 563, 565, and 567-573.

36. The method of claim 32, wherein the plant cell is from a monocot plant.

37. The method of claim 36, wherein the monocot plant is maize.

38. (canceled)

39. (canceled)

40. (canceled)

41. (canceled)

42. (canceled)

43. (canceled)

44. (canceled)

45. A method for increasing photosynthetic activity in a plant, the method comprising:
a. expressing in a regenerable plant cell a recombinant DNA construct comprising a regulatory element operably linked to a polynucleotide encoding a polypeptide comprising an amino acid sequence that is at least 90% identical to an amino acid sequence selected from the group consisting of SEQ ID NOS: 1-11, 23-31, 40-299, 563, 565, and 567-573; and
b. generating the plant, wherein the plant comprises in its genome the recombinant DNA construct.

46. The method of claim 45, wherein the regulatory element is a heterologous promoter.

47. The method of claim 45, wherein the wherein the plant exhibits increased expression of a polynucleotide that encodes a polypeptide selected from the group consisting of SEQ ID NOS: 40-222, when compared to the control plant.

48. The method of claim 45, wherein the polynucleotide encodes a polypeptide comprising an amino acid sequence that is at least 95% identical to an amino acid sequence selected from the group consisting of SEQ ID NOS: 1-11, 23-31, 40-299, 563, 565, and 567-573.

49. The method of claim 47, wherein the plant cell is from a monocot plant.

50. The method of claim 49, wherein the monocot plant is maize.

51. A method for increasing photosynthetic activity in a plant, the method comprising:
a. introducing in a regenerable plant cell a targeted genetic modification at a genomic locus that encodes a polypeptide comprising an amino acid sequence that is at least 80% identical to an amino acid sequence selected from the group consisting of SEQ ID NOS: 1-11, 23-31, 40-299, 563, 565, and 567-573; and
b. generating the plant, wherein the level and/or activity of the encoded polypeptide is increased in the plant.

52. The method of claim 51, wherein the polynucleotide encodes a polypeptide comprising an amino acid sequence that is at least 95% identical to an amino acid sequence selected from the group consisting of SEQ ID NOS: 1-11, 23-31, 40-299, 563, 565, and 567-573.

53. The method of claim 51, wherein the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute.

54. The method of claim 51, wherein the targeted genetic modification is present (a) in the coding region; (b) a non-coding region; (c) a regulatory sequence; (d) an untranslated region; or (e) any combination of (a)-(d) of the genomic locus that encodes the polypeptide.

55. The method of claim 51, wherein the plant cell is from a monocot plant.

56. The method of claim 55, wherein the monocot plant is maize.

57. (canceled)

58. (canceled)
